(19) United States
(12) Patent Application Publication
(10) Pub. No.: US 2005/0038259 A1
(43) Pub. Date: Feb. 17, 2005

(54) Hydroxyeicosenoic acid anaglogs

(76) Inventors: John R. Falck, Dallas, TX (US); Noriyuki Miyata, Toshima-ku (JP); Naoya Ono, Toshima-ku Tokyo (JP); Tomomichi Chonan, Toshima-ku (JP); Hitomi Hirano, Toshima-ku Tokyo (JP); Yoshihisa Toda, Toshima-ku (JP); Tohru Tanami, Toshima-ku Tokyo (JP); Shigeru Okuyama, Toshima-ku (JP)

Correspondence Address: SUGHRUE MION, PLLC, 2100 PENNSYLVANIA AVENUE, N.W., SUITE 800, WASHINGTON, DC, 20037, US

(21) Appl. No.: 10/489,205

(22) PCT Filed: Sep. 9, 2002
(86) PCT No.: PCT/US02/25971

Related U.S. Application Data
(60) Provisional application No. 60/318,874, filed on Sep. 14, 2001.

(51) Int. Cl. C07D 277/12; C11C 3/00

(52) U.S. Cl. 548/183; 548/255; 554/54; 554/213

(57) ABSTRACT

A hydroxyeicosenoic acid analog represented by the following Formula (I), the bond ≡ represents a cis-vinylene group or an ethynylene group; Y represents CH², O or S(O)^{p} wherein p is 0, 1 or 2; m represents an integer of 1 to 4 inclusive; n represents an integer of 0 to 3 inclusive; the sum of m and n is an integer of 3 to 7 inclusive; R¹ represents a C¹⁻⁴ alkyl group or a C³⁻⁸ cycloalkyl group; R² represents a hydrogen atom or a methyl group; R³ represents COR⁴, a nitrile group, a halogen atom, a tetrazole group or a thiazolidinedione group; R⁴ represents OR⁶, NHR⁶, N(OH)R⁶, NHSO²R⁵, glycerol or functionalized glycerols; R⁵ represents a C₁₋₁₅ alkyl group, a C₆₋₁₀ aryl group or a C₇₋₁₄ aryl group substituted with alkyl groups, halogens or amino groups; R⁶ represents a hydrogen, a C₁₋₁₀ alkyl group or a C₁₋₁₀ alkyl group substituted with a hydroxyl group, or a pharmaceutically acceptable salt or hydrate thereof. The compounds of the present invention are useful as an elastase release inhibitor.

This application is based on and claims priority from U.S. Provisional Patent Application No. 60/318,874, filed Sep. 14, 2001 which is incorporated herein by reference in its entirety for all purposes.

### TECHNICAL FIELD

This invention relates to a novel hydroxy-eicosenoic acid analog having an elastase release-inhibiting activity, a pharmaceutically acceptable salt or hydrate thereof.

The invention also relates to an elastase release-inhibiting composition which comprises as an active ingredient the hydroxyeicosenoic acid analog.

### BACKGROUND ART

Protease produced from neutrophils, one of lymphocytes, plays a main role in degrading foreign microorganisms such as bacteria or damaged cells and thus plays an important role in biophylactic reaction. Neutrophilic elastase, one of serine proteases, (hereinafter simply referred to as elastase) is abundantly released from granules of neutrophils which may develop in the case of infections or inflammatory disorders. Elastase is an enzyme capable of decomposing proteins such as elastin, collagen, proteoglycan, fibronectin, etc., which constitute stroma of in vivo connecting tissues such as lung, cartilage, vascular wall, skin, ligament and so on. Further, it has been elucidated that this enzyme may also act on other proteins or cells.

The elastase maintains homeostasis of a living body, while its action is under control by endogenous inhibitor proteins, typically, α1-protease inhibitor, α2-macroglobulin, secretory leukocyte protease inhibitor, etc. However, where a balance of elastase and endogenous inhibitor is lost by overproduction of elastase in inflammatory sites or by a lowered inhibitor level, the activity of elastase release may become uncontrollable to cause damage of tissues.

Elastase is known to be involved in pathology of certain diseases such as pulmonary emphysema, respiratory distress syndrome of adults, idiopathic pulmonary fibrosis, cystic pulmonary fibrosis, chronic interstitial pneumonia, chronic bronchitis, chronic sinopulmonary infection, diffuse panbronchiolitis, bronchiectasis, asthma, pancreatitis, nephritis, hepatic insufficiency, chronic rheumatism, arthrosclerosis, osteoarthritis, psoriasis, periodontitis, atherosclerosis, rejection against organ transplantation, premature amniorrhexis, hydroa, shock, sepsis, systemic lupus erythematosus, Crohn's disease, disseminated intravenous coagulation, cerebral infarction, cardiac disorders, ischemic reperfusion disorders observed in renal diseases, cicatrization of corneal tissues, spondylitis, and etc.

In view of the foregoing, an elastase release inhibitor is useful as a therapeutic or preventive agent for these diseases. Extensive studies have recently been made with expectation and various elastase release inhibitors have been reported. However, their activity is not quite satisfactory. Moreover, any clinically useful drug has not yet been found out as an elastase release-inhibiting agent comprising a hydroxy-eicosenoic acid analog.

### DISCLOSURE OF INVENTION

It is an object of this invention to provide a novel compound having a prominent elastase release-inhibiting activity.

It is another object of this invention to provide an elastase release-inhibiting composition which comprises the hydroxyeicosenoic acid analog or a pharmaceutically acceptable salt or hydrate thereof and pharmaceutically acceptable carrier.

### BRIEF DESCRIPTION OF THE DRAWINGS

FIG. 1 represents an effect of the compound 50 on infarct volume in rat t-MCAo model. The infarct volumes of total (closed bar), cortex (solid bar) and sub-cortex (open bar) were determined 71 hrs after reperfusion. Data are presented as mean ±SEM. *p<0.05 vs vehicle-treated group (Dunnett's test).

### DETAILED DESCRIPTION

The present inventors studied intensively to find that a novel hydroxyeicosenoic acid analog represented by the following formula shows an elastase release-inhibiting activity, upon which this invention has been completed.

More specifically, the invention is directed to a hydroxyeicosenoic acid analog represented by the following formula (I), 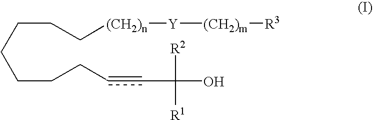
wherein
the bond [symbol] represents a cis-vinylene group or an ethynylene group;
Y represents CH₂, O or S(O)ₚ, wherein p is 0, 1 or 2;
m represents an integer of 1 to 4 inclusive and n represents an integer of 0 to 3 and the sum of m and n is an integer of 3 to 7 inclusive;
R¹ represents a C₁₋₄ alkyl group or a C₃₋₈ cycloalkyl;
R² represents a hydrogen atom, or a methyl group;
R³ represents COR⁴, a nitrile group, a halogen atom, a tetrazole group, or a thiazolidinedione group, wherein R⁴ is OR⁶, NHR⁶, N(OH)R⁶, NHSO₂R⁵ (wherein R⁵ is a C₁₋₁₅ alkyl group, a C₆₋₁₀ aryl group or a C₇₋₁₄ aryl group which is substituted with alkyl groups, halogens or amino groups and R⁶ is a hydrogen atom, a C₁₋₁₀ alkyl group or a C₁₋₁₀ alkyl group substituted with a hydroxyl group) or glycerol and functionalized glycerols (e.g., diacylglycerol and phosphoglycerides) or pharmaceutically acceptable salt or hydrate thereof. Especially preferred compounds are (R)-16-Hydroxyeicos-14-ynoic acid, (R)-17-Hydroxyheneicos-15-ynoic acid, (R)-(Z)-16-Hydroxyeicos-14-enoic acid and (R)-(Z)-15-Hydroxynonadec-13-enoic acid.

As used herein, the term “C₁₋₄ alkyl group” means a straight or branched alkyl group, which includes, for example, a methyl group, an ethyl group, a propyl group, an isopropyl group, a butyl group, an isobutyl group and a tert-butyl group.

The symbols m represents an integer of 1-4 inclusive and n represents an integer of 0-3, and the sum of m and n is 3-7 inclusive, preferably the sum being 3, 4 or 5.

As used herein, the “C₃₋₈ cycloalkyl group” includes, for example, a cyclopropyl group, a cyclobutyl group, a cyclopentyl group, a cyclohexyl group, a cycloheptyl group and a cyclooctyl group.

As used herein, the “C₁₋₁₅ alkyl group” includes, for example, a methyl group, a butyl group, a tert-butyl group, an octyl group, a decyl group, and a pentadecyl group.

As used herein, the “C₆₋₁₀ aryl” includes, for example, a phenyl group, a 1-naphthyl group and a 2-naphthyl group.

As used herein, the “C₇₋₁₄ aryl group which is substituted with alkyl groups, halogens or amino groups” includes, for example, a p-tolyl group, an o-tolyl group, a mesityl group and a m-cumenyl group, m-chlorophenyl and p-aminophenyl group.

As used herein, the term “C₁₋₁₀ alkyl group” means a straight or branched alkyl group, which includes, for example, a methyl group, an ethyl group, a butyl group, an isobutyl group, a pentyl group, a hexyl group, a heptyl group, an octyl group, a 2-methyl-1-hexyl group, a 2,4-dimethyl-1-pentyl group, a nonyl group and a decyl group.

As used herein, the term “C₁₋₁₀ alkyl group substituted with a hydroxyl group” means a straight or branched alkyl group substituted with a hydroxyl group, which includes, for example, a 2-hydroxyethyl group, a 6-hydroxyhexyl group, a 1-hydroxy-2-propyl group or a 1-hydroxy-2-methyl-2-propyl group.

As used herein, “pharmaceutically acceptable salts” includes, for example, salts with an alkali metal, e.g., sodium and potassium, salts with an alkaline earth metal, e.g., calcium and magnesium, or salts with ammonia, methylamine, dimethylamine, diethylamine, cyclopentylamine, benzylamine, piperidine, monoethanolamine, diethanolamine, triethanolamine, monomethylmonoethanolamine, toromethamine, lysine, ornithine, piperazine, benzathine, 3-aminopyridine, procaine, choline, 2-amino-4-methylpyridine, a tetraalkyl-ammonium, tris(hydroxymethyl)aminomethane and ethylenediamine.

The compounds of the formula (I) can be prepared, for example, by the processes as shown in the following Reaction Schemes.

In the Reaction Schemes, Z and Z² may be the same or different and each represents a halogen atom or a leaving group such as a methanesulfonyloxy group and a p-toluenesulfonyloxy group; R⁷ represents a protecting group for hydroxyl group, which is stable to a base, such as a trimethylsilyl group, a triethylsilyl group, tert-butyldimethylsilyl group, tert-butyldiphenylsilyl group, a methoxymethyl group, an ethoxyethyl group, a tetrahydropyranyl group, a benzyl group and a p-methoxybenzyl group; R³¹ represents CO₂H, OR⁶, CONHR⁶ or a halogen atom; R⁶¹ is the same as R⁶ excluding the hydrogen atom; R³² represents CO₂R⁶¹, OR⁶ or CONHR⁶; p1 is an integer of 1 or 2; and R¹, R², R³, R⁴, R⁵, R⁶, [symbol] , Y, m, n and p are as defined above. 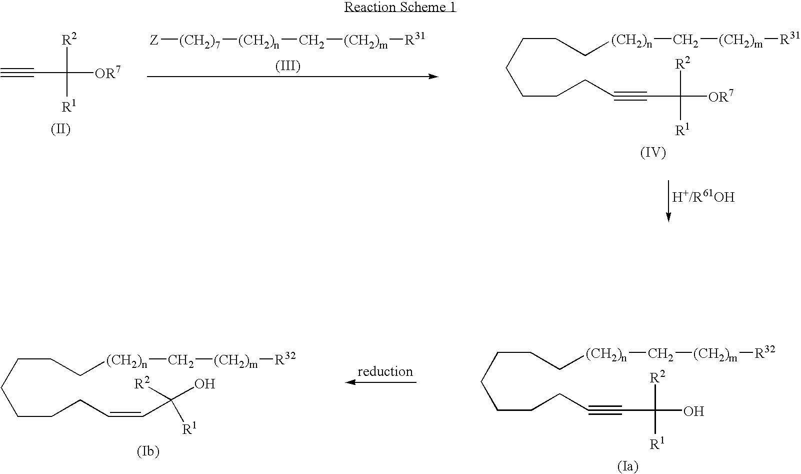 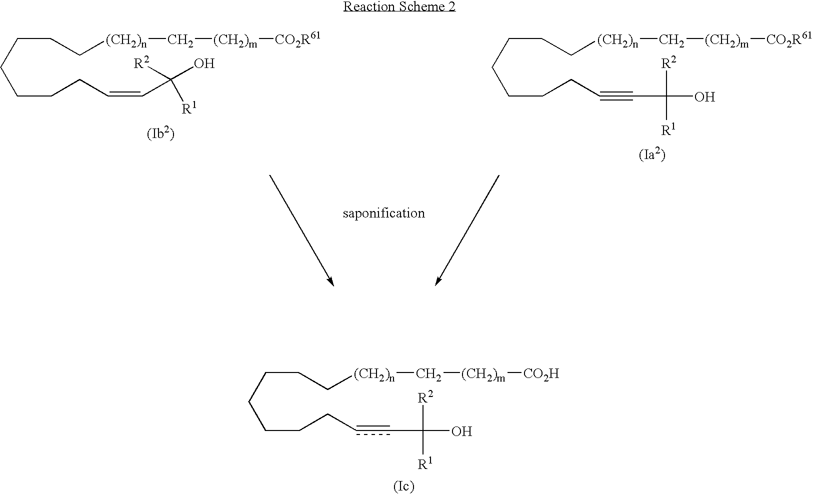
(1) A compound of the formula (II) is reacted with a compound of the formula (III) in a suitable organic solvent such as tetrahydrofuran, hexamethylphosphoric triamide, N,N′-dimethylpropyleneurea, NH₃, dimethyl sulfoxide or dimethylformamide, or a mixture thereof, in the presence of a base such as n-BuLi, LiNH₂ or NaNH₂ at a temperature of −78° C. to room temperature to give a compound of the formula (IV).
(2) A compound of the formula (IV) is treated with an organic acid such as p-toluenesulfonic acid or acetic acid, or an amine salt thereof such as pyridinium p-toluenesulfonate, or an inorganic acid such as hydrochloric acid or sulfuric acid, in a suitable organic solvent such as an alcohol solvent represented by R⁶¹OH or an ether solvent, e.g., tetrahydrofuran or diethyl ether, at a temperature of 0° C. to 60° C., preferably from room temperature to 40° C. thereby removing the protecting group for the hydroxyl group to give a compound of the formula (Ia).
(3) A compound of the formula (Ia) is reduced, for example, by a method using a Pd-containing catalyst, e.g., Pd—CaCO₃, Pd(OAc)₂ or a Ni-containing catalyst, e.g., Ni(OAc)₂ and NaBH₄ under hydrogen atmosphere, a method using Zn as a reducing agent in MeOH or AcOH and others to give a compound of the formula (Ib).
(4) A compound of the formula (Ia²) wherein R³² in the formula (Ia) is CO₂R⁶¹ or a compound of the formula (Ib²) wherein R³² in the formula (Ib) is CO₂R⁶¹ is treated with a base conventionally employed for hydrolysis such as NaOH, LiOH or KOH, in a mixed solvent of a suitable organic solvent such as an alcohol solvent, e.g., MeOH or EtOH, or a water-miscible solvent, e.g., tetrahydrofuran or dioxane, and water to give a compound of the formula (Ic) wherein R³ in the formula (I) is CO₂H. 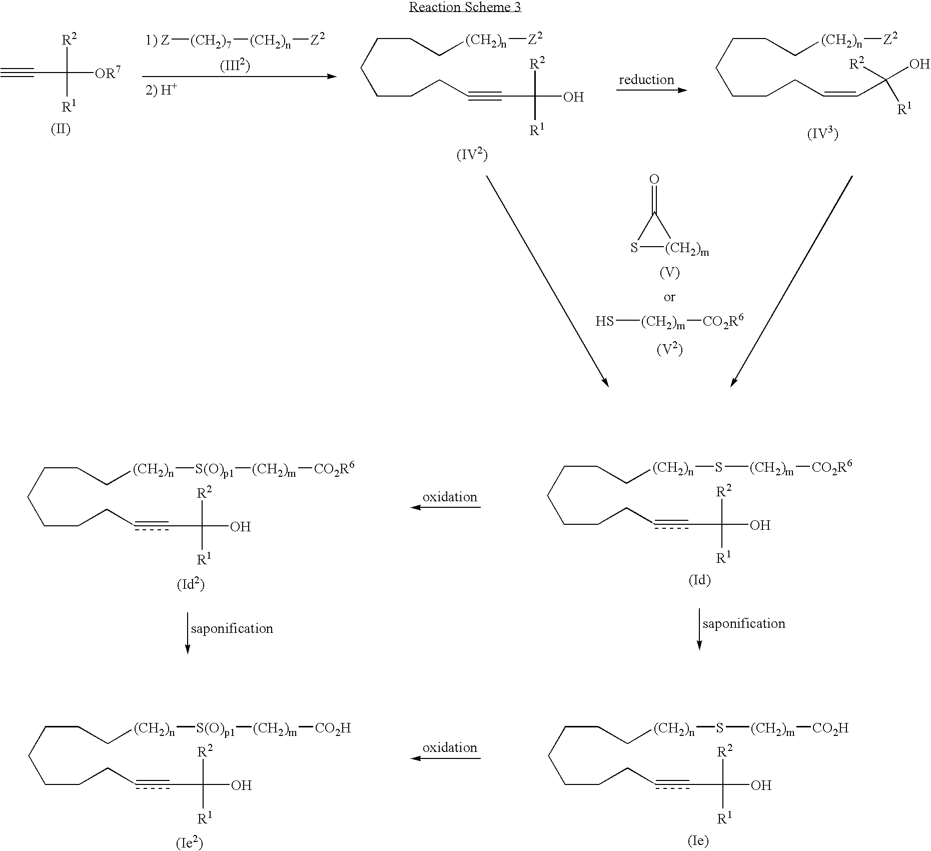
(5) A compound of the formula (II) and a compound of the formula (III²) are reacted in the same manner as in the above (1) followed by deprotection in the same manner as in the above (2) to give a compound of the formula (IV²).
(6) A compound of the formula (IV²) is reduced in the same manner as in the above (3) to give a compound of the formula (IV³).
(7) A compound of the formula (IV²) or (IV³) is reacted with a compound of the formula (V) or (V²) in a suitable organic solvent such as MeOH, EtOH, t-BuOH, acetone, dimethylformamide, tetrahydrofuran or CH₃CN, in the presence of a suitable base such as Et₃N, NaH, KH, NaHCO₃, K₂CO₃, NaOH, CaCO₃ or quaternary ammonium salt (e.g., Et₄NBr) and, where necessary, further adding NaI or the like, to give a compound of the formula (Id).
(8) A compound of the formula (Id) is hydrolyzed in the same manner as in the above (4) to give a compound of the formula (Ie).
(9) A compound of the formula (Id) or (Ie) is treated with an oxidizing agent such as NaIO₄, H₂O₂, AcOOH, m-chloroperbenzoic acid or tert-BuOOH, in a suitable organic solvent such as dichloromethane, MeOH, EtOH, diethyl ether or water, or a mixture thereof, at a temperature of −20° C. to 50° C., to give a compound of the formula (Id²) or (Ie²), respectively. A compound of the formula (Ie²) may be also prepared by hydrolyzing the compound of the formula (Id²) in the same manner as in the above (4). 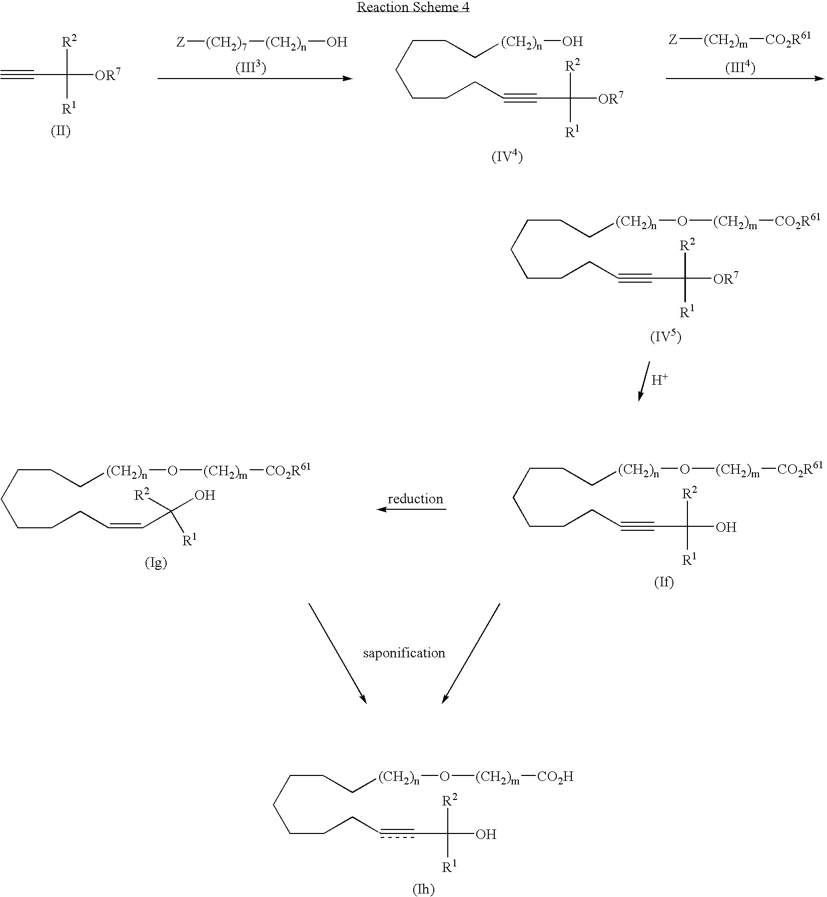 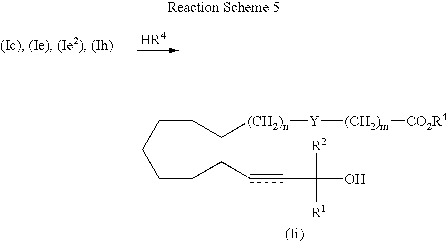
(10) A compound of the formula (II) and a compound of the formula (III³) are reacted in the same manner as in the above (1) to give a compound of the formula (IV⁴).
(11) A compound of the formula (IV⁴) is reacted with a compound of the formula (III⁴) in a suitable organic solvent such as benzene, toluene, dimethylformamide, dimethyl sulfoxide, hexamethylphosphoric triamide or CH₃CN, in the presence of a suitable base such as NaOH, KOH, NaH, KH or K₂CO₃, or Ag₂O, and where necessary, an additional agent such as n-Bu₄NI or n-Bu₄NHSO₄, to give a compound of the formula (IV⁵).
(12) A compound of the formula (IV⁵) is reacted in the same manner as in the above (2) to give a compound of the formula (If).
(13) A compound of the formula (If) is reacted in the same manner as in the above (3) to give a compound of the formula (Ig).
(14) A compound of the formula (If) or (Ig) is reacted in the same manner as in the above (4) to give a compound of the formula (Ih).
(15) A compound of the formula (Ic), (Ie), (Ie²) or (Ih) is converted into the corresponding active ester with N-hydroxy-succinimide and 1-(3-dimethylaminopropyl)-3-ethylcarbodiimide hydrochloride or N,N′-carbonyldiimidazole or the corresponding acid chloride with SOCl₂ or (COCl)₂, which is then allowed to react with HR⁴, where necessary, in the presence of a base such as 1,8-diazabicyclo[5.4.0]undec-7-ene, 1,5-diazabicyclo[4.3.0]non-5-ene or Et₃N, to give a compound of the formula (Ii). 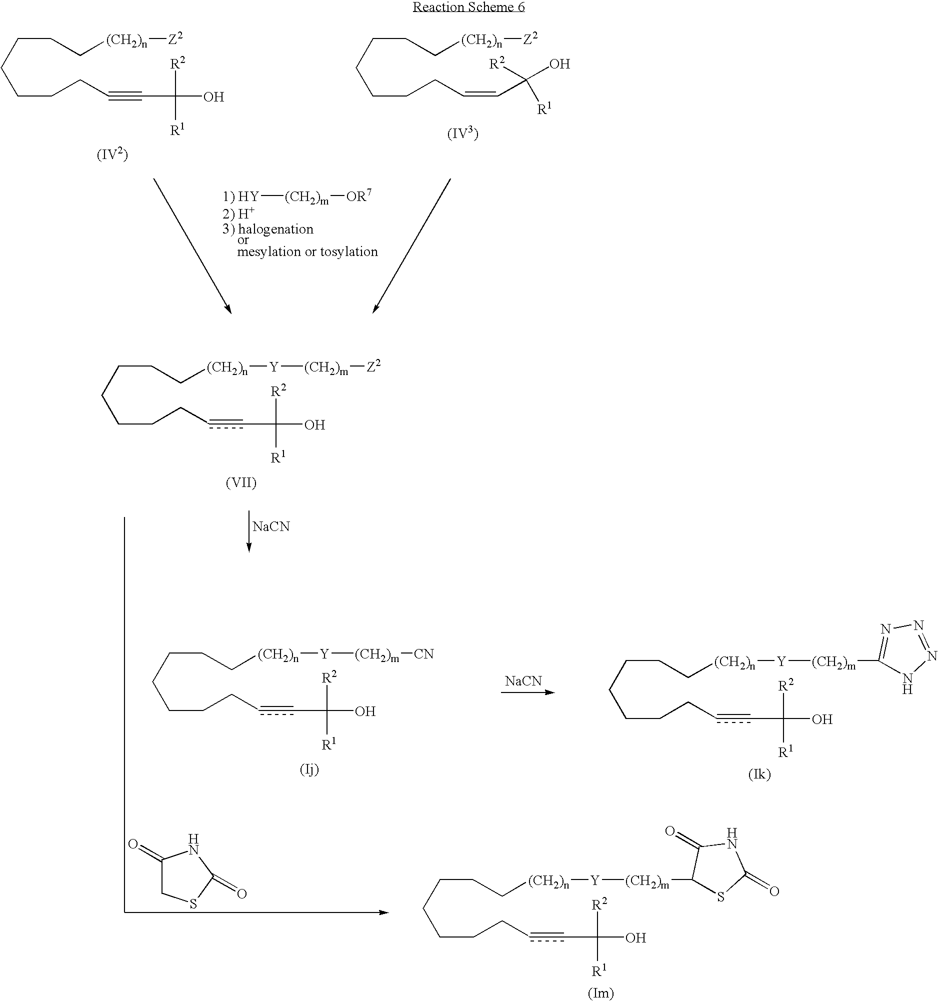
(16) A compound of the formula (IV²) or (IV³) is reacted with the compound of the formula (VI) in the same manner as in the above (7), followed by deprotection in the same manner as in the above (2) and then direct halogenation using CCl₄-PPh₃, PBr₃, CBr₄-PPh₃, I₂-PPh₃ or the like, or convertion to a leaving group using methanesulfonyl chloride, p-toluenesulfonyl chloride or the like, to give a compound of the formula (VII).
(17) A compound of the formula (VII) is reacted with a cyanation agent such as NaCN, KCN, LiCN or CuCN in a suitable organic solvent such as dimethyl sulfoxide, dimethylformamide, tetrahydrofuran, CH₃CN, toluene or benzene, or a mixed solvent thereof with water, and where necessary, in the presence of an additive such as 15-crown ether or n-Bu₄NI, to give a compound of the formula (Ij). The compound of the formula (Ij) is further reacted with an azide-forming agent such as NaN₃ or Me₃SiN₃ to give a compound of the formula (Ik).
(18) A compound of the formula (VII) is reacted with a thiazolidinedione to give a compound of the formula (Im).

The present compounds may be administered systemically or orally via oral or parenteral, such as rectal, subcutaneous, intermuscular, intravenous, transdermal and nasal/lung inhalation or percutaneous route.

They can be administered orally in the dosage form of tablets, powders, granules, fine powders, capsules, solutions, emulsions, suspensions or the like as prepared in a conventional manner. A pharmaceutical preparation for intravenous route may be in the form of aqueous or non-aqueous solutions, emulsions, suspensions, solid preparations to be used after dissolving in an injectable solvent immediately before application, or the like. The compounds of the invention may be formulated into a pharmaceutical preparation by forming an inclusion compound with α-, β- or γ-cyclodextrin or substituted cyclodextrin. Also, aqueous or non-aqueous solutions, emulsions or suspensions of the compounds may be administered, for example, via injection. A dose may be varied depending on the age, body weight and other factors of patients, and 1 ng/kg/day—1000 mg/kg/day is given to adults once a day or in several divided forms.

Representative compounds represented by the formula (I) will be illustrated below:

| Compound No. | R¹ | R² | ≡ | Y | m | n | R³ | * |
|---|---|---|---|---|---|---|---|---|
| 1 | nBu | H | C≡C | CH₂ | 4 | 3 | CO₂H | R |
| 2 | nBu | H | C≡C | CH₂ | 3 | 3 | CO₂H | R |
| 3 | nBu | H | C≡C | CH₂ | 2 | 3 | CO₂Et | R |
| 4 | nBu | H | C≡C | CH₂ | 2 | 3 | CO₂H | R |
| 5 | Me | H | C≡C | CH₂ | 2 | 3 | CO₂H | R |
| 6 | Me | H | C≡C | CH₂ | 1 | 3 | CO₂H | RS |
| 7 | Et | H | C≡C | CH₂ | 1 | 3 | CO₂Et | RS |
| 8 | Et | H | C≡C | CH₂ | 1 | 3 | CO₂H | RS |
| 9 | nPr | H | C≡C | CH₂ | 1 | 3 | CO₂Et | RS |
| 10 | nPr | H | C≡C | CH₂ | 1 | 3 | CO₂H | RS |
| 11 | nBu | H | C≡C | CH₂ | 1 | 3 | CO₂Et | R |
| 12 | nBu | H | C≡C | CH₂ | 1 | 3 | CO₂H | R |
| 13 | nBu | H | C≡C | CH₂ | 1 | 3 | CO₂H | S |
| 14 | iBu | H | C≡C | CH₂ | 1 | 3 | CO₂H | R |
| 15 | sBu | H | C≡C | CH₂ | 1 | 3 | CO₂nHex | R |
| 16 | sBu | H | C≡C | CH₂ | 1 | 3 | CO₂H | R |
| 17 | cPent | H | C≡C | CH₂ | 1 | 3 | CO₂H | R |
| 18 | cHep | H | C≡C | CH₂ | 1 | 3 | CO₂tBu | R |
| 19 | cPent | H | C≡C | CH₂ | 1 | 3 | CO₂H | R |
| 20 | cOct | H | C≡C | CH₂ | 1 | 3 | CO₂H | R |
| 21 | nBu | H | C≡C | CH₂ | 1 | 3 | CO₂Na | R |
| 22 | nPr | H | C≡C | CH₂ | 1 | 3 | CONH₂ | RS |
| 23 | nBu | H | C≡C | CH₂ | 1 | 3 | CONH₂ | RS |
| 24 | Et | Me | C≡C | CH₂ | 1 | 3 | CONHOH | RS |
| 25 | nPr | H | C≡C | CH₂ | 1 | 3 | CONHOH | R |
| 26 | nBu | H | C≡C | CH₂ | 1 | 3 | CONHOH | RS |
| 27 | nBu | H | C≡C | CH₂ | 1 | 2 | CO₂Et | R |
| 28 | nBu | H | C≡C | CH₂ | 1 | 2 | CO₂H | R |
| 29 | Et | Me | C≡C | CH₂ | 1 | 2 | CO₂H | RS |
| 30 | Me | H | C≡C | CH₂ | 1 | 2 | CO₂H | R |
| 31 | nBu | H | C≡C | CH₂ | 1 | 2 | CONH₂ | R |
| 32 | nBu | H | C≡C | CH₂ | 1 | 2 | CONHOH | R |
| 33 | nBu | H | C≡C | CH₂ | 1 | 2 | thiazolidinedione | R |
| 34 | nBu | H | C≡C | CH₂ | 1 | 3 | Cl | R |
| 35 | nBu | H | C≡C | CH₂ | 1 | 3 | OH | R |
| 36 | nBu | H | C≡C | CH₂ | 1 | 3 | tetrazole | R |
| 37 | nBu | H | C≡C | CH₂ | 1 | 3 | CN | R |
| 38 | nBu | H | C≡C | CH₂ | 3 | 3 | CN | R |
| 39 | nBu | H | C≡C | CH₂ | 2 | 3 | OH | R |
| 40 | nBu | H | C≡C | CH₂ | 2 | 3 | OMe | R |
| 41 | nBu | H | (Z)CH═CH | CH₂ | 4 | 3 | CO₂H | R |
| 42 | nPr | Me | (Z)CH═CH | CH₂ | 3 | 3 | CO₂H | RS |
| 43 | nBu | H | (Z)CH═CH | CH₂ | 3 | 3 | CO₂H | R |
| 44 | nBu | H | (Z)CH═CH | CH₂ | 2 | 3 | CO₂Et | R |
| 45 | nBu | H | (Z)CH═CH | CH₂ | 2 | 3 | CO₂iPr | R |
| 46 | nBu | H | (Z)CH═CH | CH₂ | 2 | 3 | CO₂H | R |
| 47 | nBu | H | (Z)CH═CH | CH₂ | 1 | 3 | CO₂Et | R |
| 48 | Et | H | (Z)CH═CH | CH₂ | 1 | 3 | CO₂H | RS |
| 49 | nPr | H | (Z)CH═CH | CH₂ | 1 | 3 | CO₂H | RS |
| 50 | nBu | H | (Z)CH═CH | CH₂ | 1 | 3 | CO₂H | R |
| 51 | sBu | H | (Z)CH═CH | CH₂ | 1 | 3 | CO₂H | R |
| 52 | nBu | H | (Z)CH═CH | CH₂ | 1 | 3 | CONH(CH₂)₂OH | R |
| 53 | nBu | H | (Z)CH═CH | CH₂ | 1 | 3 | CONHTs | R |
| 54 | nBu | Me | (Z)CH═CH | CH₂ | 1 | 3 | CO₂H | RS |
| 55 | nBu | H | (Z)CH═CH | CH₂ | 1 | 3 | CONHOH | R |
| 56 | nBu | H | (Z)CH═CH | CH₂ | 1 | 3 | CO₂H | S |
| 57 | nBu | H | (Z)CH═CH | CH₂ | 1 | 3 | CONH₂ | R |
| 58 | nBu | H | (Z)CH═CH | CH₂ | 1 | 3 | CONHSO₂nPentadec | R |
| 59 | nBu | H | (Z)CH═CH | CH₂ | 1 | 3 | tetrazole | R |
| 60 | nBu | H | (Z)CH═CH | CH₂ | 1 | 3 | thiazolidinedione | R |
| 61 | cPr | H | (Z)CH═CH | CH₂ | 1 | 3 | CONHSO₂nOct | R |
| 62 | cHex | H | (Z)CH═CH | CH₂ | 1 | 3 | CO₂H | R |
| 63 | iBu | H | (Z)CH═CH | CH₂ | 1 | 3 | CO₂H | RS |
| 64 | nBu | H | (Z)CH═CH | CH₂ | 1 | 2 | CO₂Et | R |
| 65 | nBu | H | (Z)CH═CH | CH₂ | 1 | 2 | CO₂H | R |
| 66 | nBu | H | (Z)CH═CH | CH₂ | 1 | 2 | CO₂Na | R |
| 67 | nBu | H | (Z)CH═CH | CH₂ | 1 | 2 | tetrazole | R |
| 68 | nBu | H | (Z)CH═CH | CH₂ | 1 | 2 | thiazolidinedione | R |
| 69 | nBu | H | (Z)CH═CH | CH₂ | 1 | 2 | CONHEt | RS |
| 70 | nBu | H | (Z)CH═CH | CH₂ | 1 | 2 | CONHMe | R |
| 71 | sBu | H | (Z)CH═CH | CH₂ | 1 | 2 | CO₂H | R |
| 72 | nPr | H | (Z)CH═CH | CH₂ | 1 | 2 | CO₂H | RS |
| 73 | nBu | H | C≡C | S | 1 | 3 | CO₂H | R |
| 74 | nBu | H | C≡C | S | 4 | 0 | CO₂Me | R |
| 75 | nBu | H | C≡C | S | 4 | 0 | CO₂H | R |
| 76 | sBu | H | C≡C | S | 4 | 0 | CO₂Me | R |
| 77 | nBu | H | C≡C | S | 3 | 0 | CO₂Me | R |
| 78 | nBu | H | C≡C | S | 3 | 0 | CO₂H | R |
| 79 | nBu | H | C≡C | S(0) | 3 | 0 | CO₂H | R |
| 80 | nBu | H | C≡C | S(0)₂ | 3 | 0 | CO₂H | R |
| 81 | nBu | H | C≡C | S | 3 | 0 | CO₂H | S |
| 82 | nBu | Me | C≡C | S | 3 | 0 | CO₂H | RS |
| 83 | cHex | H | C≡C | S | 3 | 0 | CO₂H | R |
| 84 | nBu | H | C≡C | S | 4 | 0 | CONHMs | RS |
| 85 | nBu | H | C≡C | 0 | 1 | 3 | CO₂Me | R |
| 86 | nBu | H | C≡C | 0 | 1 | 3 | CO₂H | R |
| 87 | nBu | H | C≡C | 0 | 1 | 3 | CONHTs | R |
| 88 | iBu | H | C≡C | 0 | 3 | 0 | CO₂H | RS |
| 89 | nBu | H | C≡C | 0 | 3 | 0 | CONHSO₂nOct | RS |
| 90 | nBu | H | (Z)CH═CH | S | 4 | 0 | CO₂Me | R |
| 91 | nBu | H | (Z)CH═CH | S | 4 | 0 | CO₂H | R |
| 92 | nBu | H | (Z)CH═CH | S | 1 | 3 | CO₂H | R |
| 93 | nBu | H | (Z)CH═CH | S | 3 | 0 | CO₂Me | R |
| 94 | nBu | H | (Z)CH═CH | S | 3 | 0 | CO₂H | R |
| 95 | nBu | H | (Z)CH═CH | S(0) | 3 | 0 | CO₂H | R |
| 96 | nBu | H | (Z)CH═CH | S(0)₂ | 3 | 0 | CO₂H | R |
| 97 | nBu | H | (Z)CH═CH | S | 4 | 1 | CO₂Et | R |
| 98 | nBu | H | (Z)CH═CH | S | 4 | 1 | CO₂H | R |
| 99 | nBu | H | (Z)CH═CH | S | 3 | 2 | CO₂H | R |
| 100 | nPr | H | (Z)CH═CH | S | 3 | 0 | CONH₂ | R |
| 101 | nBu | H | (Z)CH═CH | 0 | 1 | 3 | CO₂Me | R |
| 102 | nBu | H | (Z)CH═CH | 0 | 1 | 3 | CO₂H | R |
| 103 | cHep | H | (Z)CH═CH | 0 | 1 | 3 | CO₂H | R |
| 104 | cPr | H | (Z)CH═CH | 0 | 1 | 3 | CO₂H | R |
| 105 | nBu | H | (Z)CH═CH | 0 | 3 | 0 | CO₂H | R |
| 106 | iPr | H | (Z)CH═CH | 0 | 3 | 0 | CO₂H | RS |
| 107 | nBu | H | (Z)CH═CH | 0 | 4 | 0 | CONH₂ | RS |
| 108 | nBu | H | (Z)CH═CH | 0 | 3 | 0 | CONH₂ | R |
| 109 | nBu | H | C≡C | CH₂ | 1 | 3 | Br | R |
| 110 | nBu | H | (Z)CH═CH | CH₂ | 1 | 3 | Br | R |
| 111 | nBu | H | (Z)CH═CH | CH₂ | 1 | 3 | CN | R |

iPr: iso-propyl,
iBu: iso-butyl,
sBu: sec-butyl,
tBu: tert-butyl,
cPr: cyclopropyl,
cPent: cyclopentyl,
cHex: cyclohexyl,
cHep: cycloheptyl,
cOct: cyclooctyl,
nOct: n-octyl,
nPentadec: n-pentadecyl
*Asymmetric carbon atom to which R¹ and R² are attached.

The present compounds have a potent elastase release-inhibiting activity and are therefore useful for the treatment and prevention of diseases in which elastase is involved.

### BEST MODE FOR CARRYING OUT THE INVENTION

### EXAMPLES

This invention will be more specifically illustrated by way of the following Examples and Test Example.

### Example 1

### (R)-16-Hydroxyeicos-14-ynoic acid (Compound No. 12)

(1) n-BuLi (4.0 mL, 2.47M in hexane, 9.9 mmol) was added dropwise at −50° C., under argon stream, to a solution of (R)-3-tert-butyldimethylsiloxy-1-heptyne (1.02 g, 4.5 mmol), which had been prepared by a conventional silylation reaction of (R)-1-heptyn-3-ol, and 13-bromotridecanoic acid (1.32 g, 4.5 mmol) in a mixed solvent of THF (tetrahydrofuran) (20 mL) and HMPA (hexamethylphosphoric triamide) (2.5 mL). Thereafter, the temperature of the reaction solution was allowed to rise up to room temperature over about 2.5 hours and then stirred at that temperature for 2 hours. To the resulting solution was added an aqueous hydrochloric acid (150 mL, 1.0 M) and the mixture was extracted with Et₂O (100 mL×2). The organic layer was washed with brine (100 mL), dried over anhydrous magnesium sulfate and concentrated. The resulting crude product was dissolved in EtOH (22.5 mL), conc. sulfuric acid (0.5 mL) was added and then the mixture was stirred at room temperature for 3 days. To the reaction solution was added a saturated aqueous sodium bicarbonate (150 mL) and the mixture was extracted with Et₂O (100 mL×2). The resulting organic layer was washed with a saturated aqueous sodium bicarbonate (150 mL), dried over anhydrous magnesium sulfate and concentrated. The resulting crude product was purified by silica gel column chromatography to afford (R)-16-hydroxyeicos-14-ynoic acid ethyl ester (667 mg).

¹H-NMR (CDCl₃, 300 MHz) δ ppm: 0.92 (t, J=7.1 Hz, 3H), 1.20-1.75 (m, 26H), 1.25 (t, J=7.1 Hz, 3H), 2.20 (dt, J=1.9, 7.0 Hz, 2H), 2.29 (t, J=7.5 Hz, 2H), 4.12 (q, J=7.1 Hz, 2H), 4.30-4.40 (m, 1H).

IR (neat): 3436, 2928, 2855, 1737, 1466, 1375, 1180, 1102, 1036, 723 cm⁻¹.
(2) Aqueous NaOH (1.3 mL, 1.0 M, 1.3 mmol) was added at room temperature to a solution of the compound obtained in the above (1) (115 mg, 0.33 mmol) in a mixed solvent of THF (12.2 mL) and water (4.1 mL), and the mixture was stirred at room temperature for 3 days. The reaction solution was made acidic with aqueous oxalic acid (1.0 M), water (100 mL) was added and then the mixture was extracted with AcOEt (100 mL×2). The organic layer was washed with brine (100 mL), dried over anhydrous magnesium sulfate and concentrated. The resulting crude product was purified by silica gel column chromatography to afford the title compound (102 mg).

¹H-NMR (CDCl₃, 300 MHz) δ ppm: 0.92 (t, J=7.2 Hz, 3H), 1.18-1.78 (m, 26H), 2.20 (dt, J=1.8, 7.0 Hz, 2H), 2.35 (t, J=7.5 Hz, 2H), 4.30-4.38 (m, 1H).

IR (KBr): 3403, 2920, 2852, 1698, 1472, 1434, 1413, 1279, 1256, 1232, 1209, 1188, 1147, 1113, 1051, 940, 718, 602, 472, 418 cm⁻¹.

### Example 2

### (R)-(Z)-16-Hydroxyeicos-14-enoic acid (Compound No. 50)

(1) A suspension of NaBH₄ (8.0 mg, 0.21 mmol) in EtOH (1.0 mL) was added dropwise, under a hydrogen atmosphere, to a solution of Ni(OAc)₂.4H₂O (30 mg, 0.105 mmol) in EtOH (5 mL) and the mixture was stirred at room temperature for 30 minutes. To the reaction solution was added dropwise ethylenediamine (0.06 mL, 1.05 mmol) at room temperature, a solution of the compound as obtained in Example 1 (1) (370 mg, 1.05 mmol) in EtOH (2.0 mL) was then added dropwise and the mixture was stirred at room temperature for about 5 hours until absorption of hydrogen gas ceased. To the reaction solution was added Et₂O (50 mL), the mixture was stirred for 10 minutes and then filtered through a silica gel pad and concentrated. The resulting crude product was purified by silica gel column chromatography to afford (R)-(Z)-16-hydroxyeicos-14-enoic acid ethyl ester (265 mg).

¹H-NMR (CDCl₃, 300 MHz) δ ppm: 0.90 (t, J=6.8 Hz, 3H), 1.23-1.48 (m, 27H), 1.55-1.66 (m, 2H), 2.04-2.12 (m, 2H), 2.29 (t, J=7.5 Hz, 2H), 4.12 (q, J=7.2 Hz, 2H), 4.37-4.48 (m, 1H), 5.32-5.40 (m, 1H), 5.44-5.53 (m, 1H).

IR (neat): 3427, 2926, 2854, 1739, 1466, 1375, 1180, 1100, 1030, 724 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.90 (t, J=6.8 Hz, 3H), 1.23-1.49 (m, 24H), 1.54-1.70 (m, 2H), 2.04-2.12 (m, 2H), 2.35 (t, J=7.5 Hz, 2H), 4.44 (dt, J=6.4, 8.5 Hz, 1H), 5.32-5.41 (m, 1H), 5.44-5.54 (m, 1H).

IR (neat): 3369, 2925, 2845, 1712, 1466, 1412, 1384, 1281, 1119, 1003, 722 cm⁻¹.

### Example 3

### (R)-17-Hydroxyheneicos-15-ynoic acid (Compound No. 4)

(1) The reaction was carried out substantially in the same manner as in Example 1 (1), but using 14-bromotetradecanoic acid instead of 13-bromotridecanoic acid, to afford (R)-17-hydroxyheneicos-15-ynoic acid ethyl ester.

¹H-NMR (CDCl₃, 300 MHz) δ ppm: 0.92 (t, J=7.2 Hz, 3H), 1.19-1.74 (m, 31H), 2.20 (dt, J=1.9, 7.0 Hz, 2H), 2.29 (t, J=7.5 Hz, 2H), 4.12 (q, J=7.2 Hz, 2H), 4.28-4.41 (m, 1H).

IR (neat): 3436, 2927, 2855, 1737, 1466, 1375, 1180, 1104, 1036, 722 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.92 (t, J=7.2 Hz, 3H), 1.20-1.80 (m, 28H), 2.20 (dt, J=1.9, 7.0 Hz, 2H), 2.35 (t, J=7.5 Hz, 2H), 4.35 (tt, J=6.6, 1.9 Hz, 1H).

IR (KBr): 3371, 3281, 2922, 2849, 1702, 1465, 1438, 1412, 1316, 1274, 1228, 1206, 1188, 1150, 1111, 1051, 1012, 889, 725, 491 cm⁻¹.

### Example 4

### (R)-(Z)-17-Hydroxyheneicos-15-enoic acid (Compound No. 46)

(1) Using the compound obtained in Example 3 (1), the reaction was carried out in the same manner as in Example 2 (1) to afford (R)-(Z)-17-hydroxyheneicos-15-enoic acid ethyl ester.

¹H-NMR (CDCl₃, 300 MHz) δ ppm: 0.90 (t, J=6.8 Hz, 3H), 1.18-1.70 (m, 31H), 1.98-2.18 (m, 2H), 2.29 (t, J=7.5 Hz, 2H), 4.12 (q, J=7.2 Hz, 2H), 4.37-4.48 (m, 1H), 5.32-5.54 (m, 2H).

IR (neat): 3428, 2925, 2854, 2360, 1739, 1466, 1374, 1180, 1100, 1031, 723, 430 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.90 (t, J=6.8 Hz, 3H), 1.19-1.72 (m, 28H), 1.95-2.16 (m, 2H), 2.35 (t, J=7.5 Hz, 2H), 4.38-4.49 (m, 1H), 5.30-5.55 (m, 2H).

IR (neat): 3400, 2925, 2854, 1712, 1466, 1412, 1200, 1002, 970, 723, 430 cm⁻¹.

### Example 5

### (R)-15-Hydroxynonadec-13-ynoic acid (Compound No. 28)

(1) The reaction was carried out substantially in the same manner as in Example 1 (1), but using 12-bromododecanoic acid instead of 13-bromotridecanoic acid, to afford (R)-15-hydroxynonadec-13-ynoic acid ethyl ester.

¹H-NMR (CDCl₃, 300 MHz) δ ppm: 0.92 (t, J=7.2 Hz, 3H), 1.19-1.79 (m, 27H), 2.20 (dt, J=2.0, 7.0 Hz, 2H), 2.29 (t, J=7.5 Hz, 2H), 4.12 (q, J=7.2 Hz, 2H), 4.29-4.40 (m, 1H).

IR (neat): 3436, 2929, 2856, 2361, 1737, 1466, 1375, 1180, 1100, 1036, 722 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.92 (t, J=7.2 Hz, 3H), 1.20-1.80 (m, 24H), 2.20 (dt, J=2.0, 7.0 Hz, 2H), 2.35 (t, J=7.5 Hz, 2H), 4.35 (tt, J=6.5, 2.0 Hz, 1H).

IR (KBr): 3373, 3279, 2922, 2850, 1707, 1464, 1414, 1330, 1288, 1264, 1236, 1210, 1190, 1150, 1108, 1051, 1012, 962, 888, 726, 588 cm⁻¹.

### Example 6

### (R)-(Z)-15-Hydroxynonadec-13-enoic acid (Compound No. 65)

(1) Using the compound obtained in Example 5 (1), the reaction was carried out in the same manner as in Example 2 (1) to afford (R)-(Z)-15-hydroxynonadec-13-enoic acid ethyl ester.

¹H-NMR (CDCl₃, 300 MHz) δ ppm: 0.85-0.98 (m, 3H), 1.20-1.68 (m, 27H), 1.97-2.16 (m, 2H), 2.29 (t, J=7.5 Hz, 2H), 4.12 (q, J=7.2 Hz, 2H), 4.43 (dt, J=8.5, 6.3 Hz, 1H), 5.31-5.55 (m, 2H).

IR (neat): 3426, 2927, 2855, 1740, 1466, 1375, 1248, 1181, 1099, 1030, 724 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.91 (t, J=6.8 Hz, 3H), 1.16-1.70 (m, 24H), 1.97-2.17 (m, 2H), 2.35 (t, J=7.5 Hz, 2H), 4.38-4.49 (m, 1H), 5.30-5.54 (m, 2H).

IR (neat): 3368, 2925, 2854, 1712, 1466, 1413, 1275, 1100, 1002, 724 cm⁻¹.

### Example 7

### (RS)-(Z)-16-Hydroxy-16-methyleicos-14-enoic acid (Compound No. 54)

(1) The reaction was carried out substantially in the same manner as in Example 1 (1), but using (RS)-3-tert-butyldimethylsiloxy-3-methyl-1-heptyne instead of (R)-3-tert-butyldimethylsiloxy-1-heptyne to afford (RS)-16-hydroxy-16-methyleicos-14-ynoic acid ethyl ester, and then the reaction was carried out in the same manner as in Example 2 (1) to afford (RS)-(Z)-16-hydroxy-16-methyleicos-14-enoic acid ethyl ester.

¹H-NMR (CDCl₃, 300 MHz) δ ppm: 0.91 (t, J=7.0 Hz, 3H), 1.20-1.68 (m, 32H), 2.24-2.35 (m, 4H), 4.12 (q, J=7.2 Hz, 2H), 5.28-5.42 (m, 2H).

IR (neat): 3436, 2926, 2854, 2361, 1739, 1644, 1466, 1372, 1303, 1180, 1101, 1034, 942, 724 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.91 (t, J=7.0 Hz, 3H), 1.19-1.70 (m, 29H), 2.25-2.39 (m, 4H), 5.28-5.41 (m, 2H).

IR (neat): 3400, 2926, 2854, 1712, 1466, 1412, 1371, 1223, 1048, 940, 724 cm⁻¹.

### Example 8

### (RS)-(Z)-16-Hydroxy-18-methylnonadec-14-enoic acid (Compound No. 63)

(1) The reaction was carried out substantially in the same manner as in Example 1 (1), but using (RS)-3-tert-butyldimethylsiloxy-5-methyl-1-hexyne instead of (R)-3-tert-butyldimethylsiloxy-1-heptyne to afford (RS)-16-hydroxy-18-methylnonadec-14-ynoic acid ethyl ester, and then the reaction was carried out in the same manner as in Example 2 (1) to afford (RS)-(Z)-16-hydroxy-18-methylnonadec-14-enoic acid ethyl ester.

¹H-NMR (CDCl₃, 300 MHz) δ ppm: 0.92 (d, J=6.5 Hz, 3H), 0.94 (d, J=6.5 Hz, 3H), 1.18-1.80 (m, 26H), 2.02-2.15 (m, 2H), 2.29 (t, J=7.5 Hz, 2H), 4.12 (q, J=7.2 Hz, 2H), 4.47-4.56 (m, 1H), 5.31-5.57 (m, 2H).

IR (neat): 3436, 2926, 2854, 1739, 1466, 1369, 1180, 1034, 722 cm⁻¹.
(2) Using the compound obtained in the above (1), the reaction was carried out in the same manner as in Example 1 (2) to afford the title compound.

¹H-NMR (CDCl₃, 300 Mz) δ ppm: 0.92 (d, J=6.7 Hz, 3H), 0.94 (d, J=6.5 Hz, 3H), 1.19-1.77 (m, 23H), 2.00-2.19 (m, 2H), 2.35 (t, J=7.5 Hz, 2H), 4.47-4.57 (m, 1H), 5.30-5.40 (m, 1H), 5.42-5.52 (m, 1H).

IR (KBr): 3370, 2924, 2852, 1714, 1472, 1384, 1370, 1350, 1318, 1277, 1259, 1236, 1210, 1104, 1081, 1009, 994, 974, 823, 751, 720, 629, 556, 460 cm⁻¹.

### Example 9

### (RS)-16-Hydroxynonadec-14-ynoic acid (Compound No. 10)

(1) The reaction was carried out substantially in the same manner as in Example 1 (1), but using (RS)-3-tert-butyldimethylsiloxy-1-hexyne instead of (R)-3-tert-butyldimethylsiloxy-1-heptyne, to afford (RS)-16-hydroxynonadec-14-ynoic acid ethyl ester.

¹H-NMR (CDCl₃, 300 MHz) δ ppm: 0.91-0.99 (m, 3H), 1.20-1.78 (m, 27H), 2.20 (dt, J=2.0, 7.1 Hz, 2H), 2.29 (t, J=7.5 Hz, 2H), 4.12 (q, J=7.1 Hz, 2H), 4.31-4.43 (m, 1H).

IR (neat): 3448, 2929, 2855, 1737, 1466, 1374, 1245, 1180, 1101, 1029, 854, 723 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.95 (t, J=7.3 Hz, 3H), 1.22-1.73 (m, 24H), 2.20 (dt, J=1.9, 7.0 Hz, 2H), 2.35 (t, J=7.5 Hz, 2H), 4.36 (tt, J=6.6, 1.9 Hz, 1H).

IR (KBr): 3358, 2920, 2852, 1698, 1472, 1413, 1320, 1296, 1254, 1243, 1230, 1207, 1188, 1150, 1106, 1067, 1027, 942, 718, 474, 416 cm⁻¹.

### Example 10

### (RS)-16-Hydroxyoctadec-14-ynoic acid (Compound No. 8)

(1) The reaction was carried out substantially in the same manner as in Example 1 (1), but using (RS)-3-tert-butyldimethylsiloxy-1-pentyne instead of (R)-3-tert-butyldimethylsiloxy-1-heptyne, to afford (RS)-16-hydroxyoctadec-14-ynoic acid ethyl ester.

¹H-NMR (CDCl₃, 300 MHz) δ ppm: 1.00 (t, J=7.4 Hz, 3H), 1.18-1.78 (m, 25H), 2.20 (dt, J=1.9, 7.0 Hz, 2H), 2.29 (t, J=7.5 Hz, 2H), 4.12 (q, J=7.2 Hz, 2H), 4.26-4.36 (m, 1H).

IR (neat): 3436, 2928, 2854, 1737, 1465, 1374, 1180, 1099, 1035, 965, 722 cm⁻¹.
(2) Using the compound obtained in the above (1), the reaction was carried out in the same manner as in Example 1 (2) to afford the title compound.

¹H-NMR (CDCl₃, 300 MHz) δ ppm: 1.00 (t, J=7.4 Hz, 3H), 1.20-1.75 (m, 22H), 2.20 (dt, J=2.0, 7.0 Hz, 2H), 2.35 (t, J=7.5 Hz, 2H), 4.31 (tt, J=6.4, 1.9 Hz, 1H).

IR (KBr): 3357, 2921, 2852, 1698, 1472, 1439, 1413, 1341, 1324, 1279, 1256, 1232, 1209, 1188, 1148, 1088, 1072, 1035, 1007, 965, 718, 625 cm⁻¹.

### Example 11

### 4-((R)-10-Hydroxytetradec-8-ynylsulfanyl)butyric acid (Compound No. 78)

(1) n-BuLi (19.7 mL, 2.47 M in hexane, 48.7 mmol) was added dropwise at 0° C., under argon stream, to a solution of (R)-3-tert-butyldimethylsiloxy-1-heptyne (10.0 g, 44.3 mmol) in THF (179 mL). Thereafter, the reaction solution was stirred at that temperature for 30 minutes. The reaction solution was cooled to −40° C., to which a solution of 1,7-dibromoheptane (22.9 g, 88.6 mmol) in DMPU (N,N′-dimethylpropyleneurea) (22.4 mL) was added dropwise, and the temperature of the reaction solution was allowed to rise up to room temperature over about 2 hours and then stirred at that temperature for 2 hours. To the resulting solution was added a saturated aqueous ammonium chloride solution (500 mL) and the mixture was extracted with hexane (300 mL×2). The organic layer was washed with brine (500 mL), dried over anhydrous magnesium sulfate and concentrated. The resulting crude product was purified by distillation to afford ((R)-10-bromo-1-butyldec-2-ynyloxy)-tert-butyldimethylsilane (12.6 g).

¹H-NMR (CDCl₃, 300 MHz) δ ppm: 0.10 (s, 3H), 0.12 (s, 3H), 0.84-0.96 (m, 3H), 0.91 (s, 9H), 1.24-1.68 (m, 14H), 1.80-1.92 (m, 2H), 2.19 (dt, J=1.9, 6.9 Hz, 2H), 3.41 (t, J=6.4 Hz, 2H), 4.32 (tt, J=6.5, 1.9 Hz, 1H).

IR (neat): 2930, 2858, 2233, 1463, 1407, 1389, 1361, 1341, 1251, 1217, 1152, 1110, 1083, 1006, 938, 837, 778, 725, 667, 565 cm⁻¹.
(2) Aqueous HCl (0.5 mL, 1.0 M, 1.3 mmol) was added at room temperature to a solution of the compound obtained in the above (1) (910 mg, 2.24 mmol) in MeOH (15 mL), and the mixture was stirred at room temperature for one hour. To the reaction solution was added a saturated aqueous sodium bicarbonate solution (100 mL) and then the mixture was extracted with AcOEt (100 mL×2). The organic layer was washed with brine (100 mL), dried over anhydrous magnesium sulfate and concentrated. The resulting crude product was purified by silica gel column chromatography to afford (R)-14-bromotetradec-6-yn-5-ol (628 mg).

¹H-NMR (CDCl₃, 300 MHz) δ ppm: 0.88-0.96 (m, 3H), 1.22-1.77 (m, 14H), 1.79-1.93 (m, 2H), 2.21 (dt, J=2.0, 6.9 Hz, 2H), 3.41 (t, J=6.8 Hz, 2H), 4.29-4.40 (m, 1H).

IR (neat): 3368, 2930, 2858, 2231, 1465, 1379, 1333, 1250, 1148, 1104, 1038, 1008, 876, 726, 646, 563 cm⁻¹.
(3) NaOMe (79 mg, 1.47 mmol) was added under argon stream to a solution of the compound obtained in the above (2) (250 mg, 0.864 mmol) in MeOH (6 mL), to which a solution of γ-thiobutyrolactone (132 mg, 1.30 mmol) in MeOH (3 mL) was added dropwise, then NaI (15 mg) was added and the mixture was stirred at room temperature for 14 hours and then at 45° C. for one hour. The reaction solution was cooled to room temperature, a saturated aqueous ammonium chloride solution (50 mL) was added and then the mixture was extracted with Et₂O (50 mL×2). The organic layer was washed with brine (50 mL), dried over anhydrous magnesium sulfate and concentrated. The resulting crude product was purified by silica gel column chromatography to afford 4-((R)-10-hydroxytetradec-8-ynylsulfanyl)butyric acid methyl ester (0.23 g).

¹H-NMR (CDCl₃, 300 MHz) δ ppm: 0.92 (t, J=7.1 Hz, 3H), 1.23-1.77 (m, 16H), 1.85-1.97 (m, 2H), 2.20 (dt, J=1.9, 7.0 Hz, 2H), 2.41-2.59 (m, 6H), 3.68 (s, 3H), 4.35 (tt, J=6.6, 1.9 Hz, 1H).

IR (neat): 3453, 2930, 2858, 2230, 1740, 1437, 1366, 1315, 1212, 1175, 1145, 1037, 1008, 888, 727 cm⁻¹.
(4) Using the compound obtained in the above (3), the reaction was carried out in the same manner as in Example 1 (2) to afford the title compound.

¹H-NMR (CDCl₃, 300 MHz) δ ppm: 0.92 (t, J=7.1 Hz, 3H), 1.22-1.75 (m, 18H), 1.86-1.98 (m, 2H), 2.21 (dt, J=1.9, 6.9 Hz, 2H), 2.51 (t, J=7.2 Hz, 4H), 2.57 (t, J=7.2 Hz, 2H), 4.36 (tt, J=6.5, 1.9 Hz, 1H).

IR (neat): 3340, 2930, 2858, 2231, 1708, 1456, 1293, 1236, 1147, 1036, 1003, 889, 728 cm⁻¹.

### Example 12

### 4-((R)-(Z)-10-Hydroxytetradec-8-enylsulfanyl)butyric acid (Compound No. 94)

(1) Using the compound obtained in Example 11 (2), the reaction was carried out in the same manner as in Example 2 (1) to afford (R)-(Z)-14-bromotetradec-6-en-5-ol.

¹H-NMR (CDCl₃, 300 MHz) δ ppm: 0.84-0.96 (m, 3H), 1.20-1.67 (m, 14H), 1.79-1.92 (m, 2H), 1.98-2.16 (m, 2H), 3.41 (t, J=6.8 Hz, 2H), 4.37-4.47 (m, 1H), 5.32-5.54 (m, 2H).

IR (neat): 335i, 3006, 2930, 2856, 1656, 1466, 1378, 1252, 1121, 1007, 878, 727, 646, 564 cm⁻¹.
(2) The reaction was carried out substantially in the same manner as in Example 11 (3), but using the compound obtained in the above (1) instead of (R)-14-bromotetradec-6-yn-5-ol, to afford 4-((R)-(Z)-10-hydroxytetradec-8-enylsulfanyl)butyric acid methyl ester.

¹H-NMR (CDCl₃, 300 MHz) δ ppm: 0.91 (t, J=6.9 Hz, 3H), 1.20-1.68 (m, 16H), 1.85-2.18 (m, 4H), 1.98-2.18 (m, 2H), 2.40-2.60 (m, 6H), 3.68 (s, 3H), 4.37-4.58 (m, 1H), 5.31-5.53 (m, 2H).

IR (neat): 3436, 3004, 2928, 2855, 1740, 1438, 1366, 1314, 1211, 1174, 1140, 1006, 887, 749 cm⁻¹.
(3) Using the compound obtained in the above (2), the reaction was carried out in the same manner as in Example 1 (2) to afford the title compound.

¹H-NMR (CDCl₃, 300 MHz) δ ppm: 0.86-0.95 (m, 3H), 1.21-1.67 (m, 16H), 1.85-2.21 (m, 4H), 2.50 (2t, J=7.2 Hz, 4H), 2.57 (t, J=7.2 Hz, 2H), 4.40-4.50 (m, 1H), 5.31-5.54 (m, 2H).

IR (neat): 3368, 2929, 2856, 1708, 1457, 1293, 1235, 1138, 1000, 753 cm⁻¹.

### Example 13

### 5-((R)-10-Hydroxytetradec-8-ynylsulfanyl)pentanoic acid (Compound No. 75)

(1) The reaction was carried out substantially in the same manner as in Example 11 (3), but using δ-thiovalerolactone instead of γ-thiobutyrolactone, to afford 5-((R)-10-hydroxytetradec-8-ynylsulfanyl)pentanoic acid methyl ester.

¹H-NMR (CDCl₃, 300 MHz) δ ppm: 0.92 (t, J=7.1 Hz, 3H), 1.22-1.80 (m, 20H), 2.21 (dt, J=1.9, 7.0 Hz, 2H), 2.34 (t, J=7.3 Hz, 2H), 2.50 (t, J=7.1 Hz, 2H), 2.52 (t, J=7.2 Hz, 2H), 3.68 (s, 3H), 4.30-4.40 (m, 1H).

IR (neat): 3436, 2931, 2858, 2230, 1740, 1459, 1437, 1378, 1271, 1206, 1174, 1039, 888, 729, 504 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.92 (t, J=7.1 Hz, 3H), 1.22-1.82 (m, 20H), 2.21 (dt, J=2.0, 6.9 Hz, 2H), 2.39 (t, J=7.2 Hz, 2H), 2.51 (t, J=7.1 Hz, 2H), 2.53 (t, J=7.1 Hz, 2H), 4.35 (tt, J=6.5, 2.0 Hz, 1H).

IR (neat): 3350, 2930, 2858, 1712, 1708, 1460, 1282, 1229, 1149, 1037, 1004, 892, 727 cm⁻¹.

### Example 14

### 5-((R)-(Z)-10-hydroxytetradec-8-enylsulfanyl)pentanoic acid (Compound No. 91)

(1) The reaction was carried out substantially in the same manner as in Example 11 (3), but using the compound obtained in Example 12 (1) and δ-thiovalerolactone instead of (R)-14-bromotetradec-6-yn-5-ol and γ-thiobutyrolactone, respectively, to afford 5-((R)-(Z)-10-hydroxytetradec-8-enylsulfanyl)pentanoic acid methyl ester.

¹H-NMR (CDCl₃, 300 MHz) δ ppm: 0.86-0.95 (m, 3H), 1.21-1.79 (m, 20H), 1.98-2.18 (m, 2H), 2.34 (t, J=7.3 Hz, 2H), 2.50 (t, J=7.3 Hz, 2H), 2.52 (t, J=7.1 Hz, 2H), 3.67 (s, 3H), 4.37-4.47 (m, 1H), 5.32-5.53 (m, 2H).

IR (neat): 3436, 2928, 2855, 2360, 2343, 1740, 1437, 1384, 1271, 1205, 1174, 1009, 886, 750, 669 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.86-0.95 (m, 3H), 1.21-1.81 (m, 20H), 1.97-2.20 (m, 2H), 2.38 (t, J=7.2 Hz, 2H), 2.44-2.58 (m, 4H), 4.44 (dt, J=8.2, 6.6 Hz, 1H), 5.31-5.54 (m, 2H).

IR (neat): 3367, 3006, 2930, 2855, 1712, 1708, 1461, 1418, 1278, 1228, 1124, 1001, 897, 752 cm⁻¹.

### Example 15

### 4-((R)-10-hydroxytetradec-8-yne-1-sulfonyl)butyric acid (Compound No. 80)

m-Chloroperbenzoic acid (35 mg, 0.274 mmol) was added at room temperature to a solution of the compound obtained in Example 11 (30 mg, 0.0913 mmol) in CHCl₃ (3 mL), and the mixture was stirred at room temperature for 4 hours. To the reaction solution was added a saturated aqueous sodium thiosulfate solution (30 mL) and then the mixture was extracted with AcOEt (30 mL×2). The organic layer was washed with brine (30 mL), dried over anhydrous magnesium sulfate and concentrated. The resulting crude product was purified by silica gel column chromatography to afford the title compound (17 mg).

¹H-NMR (CDCl₃, 300 MHz) δ ppm: 0.92 (t, J=7.1 Hz, 3H), 1.21-1.92 (m, 16H), 2.10-2.27 (m, 4H), 2.60 (t, J=6.7 Hz, 2H), 2.94-3.13 (m, 4H), 4.28-4.46 (m, 1H).

IR (KBr): 3485, 3370, 2932, 2860, 1692, 1470, 1446, 1420, 1328, 1274, 1242, 1217, 1200, 1124, 1083, 1056, 1016, 912, 776, 750, 728, 613, 575, 510, 473, 420 cm⁻¹.

### Example 16

### 4-((R)-10-hydroxytetradec-8-yne-1-sulfinyl)butyric acid (Compound No. 79)

A solution of NaIO₄ (74 mg, 0.347 mmol) in water (0.9 mL) was added at room temperature to a solution of the compound obtained in Example 11 (30 mg, 0.0913 mmol) in MeOH (2.3 mL), and the mixture was stirred at room temperature for 4 hours. To the reaction solution was added brine (30 mL) and then the mixture was extracted with AcOEt (30 mL×2). The organic layer was washed with brine (30 mL), dried over anhydrous magnesium sulfate and concentrated. The resulting crude product was purified by silica gel column chromatography to afford the title compound (28 mg).

¹H-NMR (CDCl₃, 300 MHz) δ ppm: 0.92 (t, J=7.1 Hz, 3H), 1.29-1.86 (m, 16H), 2.08-2.26 (m, 4H), 2.46-2.96 (m, 6H), 4.30-4.40 (m, 1H).

IR (neat): 3368, 2933, 2859, 1724, 1456, 1412, 1291, 1225, 1144, 1034, 1003, 847, 727 cm⁻¹.

### Example 17

### (RS)-16-Hydroxyeicos-14-ynamide (Compound No. 23)

(1) A solution of acetonitrile (0.263 mL, 5.0 mmol) in THF (5 mL) was cooled to −65° C. and then n-BuLi (2.23 mL, 2.46 M in hexane, 5.5 mmol) was added dropwise, while stirring, under argon stream. Thereafter, the reaction solution was stirred at that temperature for one hour. The reaction solution was added dropwise to a solution of 1,11-dibromoundecane (3.14 g, 10 mmol) in THF (10 mL) at 0° C. over 10 minutes. The mixture was stirred at room temperature for 15 minutes. To the resulting solution were added water (10 mL) and ethyl acetate (30 mL) to separate the organic layer. It was dried over anhydrous magnesium sulfate and concentrated. The resulting crude product was purified by column chromatography to afford 13-bromotridecanitrile (800 mg).

¹H-NMR (CDCl₃, 200 MHz) δ ppm: 1.20-1.96 (m, 20H), 2.35 (t, J=7.0 Hz, 2H), 3.43 (t, J=6.8 Hz, 2H).

IR (neat): 3400, 2927, 2854, 2246, 1636, 1466, 1384, 1251, 1068, 722, 644, 562 cm⁻¹.
(2) A solution of the compound obtained in the above (1) (800 mg) in 70% aqueous sulfuric acid (0.5 mL) was stirred under heating at 70° C. for 2 hours under argon stream. The reaction solution was cooled to room temperature, ice-water (30 mL) was added and the crude crystalline substance thus separated was filtered off. The substance was dissolved in ethyl acetate (100 mL), neutralized with an aqueous sodium hydroxide solution (2.0M) and then extracted. The organic layer was washed with brine (100 mL), dried over anhydrous magnesium sulfate and concentrated. The resulting crystal was dried under reduced pressure to afford 13-bromotridecanamide (790 mg).

¹H-NMR (CDCl₃, 300 MHz) δ ppm: 1.20-1.71 (m, 18H), 1.79-1.91 (m, 2H), 2.22 (t, J=7.6 Hz, 2H), 3.41 (t, J=6.9 Hz, 2H), 5.34 (bs, 2H).

IR (KBr): 3395, 3191, 2922, 2851, 1647, 1471, 1420, 1330, 1281, 1254, 1228, 1204, 1123, 801, 721, 648, 565, 520, 472, 421 cm⁻¹.
(3) The reaction was carried out substantially in the same manner as in Example 1 (1), using the compound obtained in the above (2) and (RS)-3-tert-butyldimethylsiloxy-1-heptyne instead of 13-bromotridecanoic acid and (R)-3-tert-butyldimethylsiloxy-1-heptyne, respectively, to afford the title compound.

¹H-NMR (CDCl₃, 300 MHz) δ ppm: 0.92 (t, J=7.1 Hz, 3H), 1.20-1.80 (m, 26H), 2.16-2.27 (m, 4H), 4.35 (tt, J=6.5, 1.9 Hz, 1H), 5.28 (bs, 1H), 5.38 (bs, 1H).

IR (KBr): 3360, 3188, 2920, 2850, 1663, 1633, 1472, 1426, 1411, 1334, 1268, 1241, 1216, 1191, 1139, 1105, 1041, 882, 811, 721, 641, 530 cm⁻¹.

### Example 18

### (RS)-16-Hydroxynonadec-14-ynamide (Compound No. 22)

The reaction was carried out substantially in the same manner as in Example 1 (1), but using the compound obtained in Example 17 (2) and (RS)-3-tert-butyldimethylsiloxy-1-hexyne instead of 13-bromotridecanoic acid and (R)-3-tert-butyldimethylsiloxy-1-heptyne, respectively, to afford the title compound.

¹H-NMR (CDCl₃, 300 MHz) δ ppm: 0.95 (t, J=7.2 Hz, 3H), 1.20-1.71 (m, 24H), 2.16-2.26 (m, 4H), 4.32-4.40 (m, 1H), 5.10-5.45 (m, 2H).

IR (KBr): 3359, 3187, 2920, 2850, 1662, 1633, 1471, 1426, 1412, 1334, 1316, 1242, 1216, 1139, 1103, 1066, 1027, 946, 880, 814, 704, 643, 530 cm⁻¹.

### Example 19

### (R)-(Z)-16-Hydroxy-16-cyclohexylhexadec-14-enoic acid (Compound No. 62)

(1) The reaction was carried out substantially in the same manner as in Example 1 (1), but using (R)-3-tert-butyl-dimethylsiloxy-3-cyclohexyl-1-propyne instead of (R)-3-tert-butyldimethylsiloxy-1-heptyne to afford (R)-16-hydroxy-16-cyclohexylhexadec-14-ynoic acid ethyl ester, and then the reaction was carried out in the same manner as in Example 2 (1) to afford (R)-(Z)-16-hydroxy-16-cyclohexyl-hexadec-14-enoic acid ethyl ester.

¹H-NMR (CDCl₃, 300 MHz) δ ppm: 0.84-2.16 (m, 36H), 2.29 (t, J=7.5 Hz, 2H), 4.08-4.18 (m, 3H), 5.32-5.42 (m, 1H), 5.47-5.59 (m, 1H).

IR (neat): 3400, 2924, 2853, 1739, 1450, 1373, 1183, 1100, 1031, 973, 892, 722 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.84-1.43 (m, 24H), 1.57-1.81 (m, 6H), 1.86-2.17 (m, 3H), 2.35 (t, J=7.4 Hz, 2H), 4.15 (dd, J=9.2, 7.3 Hz, 1H), 5.32-5.42 (m, 1H), 5.48-5.59 (m, 1H).

IR (KBr): 3290, 2924, 2850, 1702, 1467, 1449, 1383, 1288, 1262, 1234, 1184, 1105, 1083, 1058, 1002, 929, 802, 729, 640, 572, 468, 444, 432, 418 cm⁻¹.

### Example 20

### (RS)-(Z)-16-Hydroxynonadec-14-enoic acid (Compound No. 49)

(1) Using the compound obtained in Example 9 (1), the reaction was carried out in the same manner as in Example 2 (1) to afford (RS)-(Z)-16-hydroxynonadec-14-enoic acid ethyl ester.

¹H-NMR (CDCl₃, 300 MHz) δ ppm: 0.93 (t, J=7.1 Hz, 3H), 1.18-1.68 (m, 27H), 2.00-2.16 (m, 2H), 2.29 (t, J=7.5 Hz, 2H), 4.12 (q, J=7.2 Hz, 2H), 4.40-4.49 (m, 1H), 5.31-5.45 (m, 2H).

IR (neat): 3400, 2925, 2854, 2361, 1737, 1646, 1465, 1384, 1318, 1179, 1098, 1026, 757 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.93 (t, J=7.2 Hz, 3H), 1.19-1.69 (m, 24H), 1.98-2.16 (m, 2H), 2.35 (t, J=7.5 Hz, 2H), 4.40-4.49 (m, 1H), 5.31-5.40 (m, 1H), 5.42-5.54 (m, 1H).

IR (KBr): 3389, 3011, 2957, 2920, 2851, 1718, 1464, 1435, 1324, 1305, 1282, 1260, 1230, 1207, 1188, 1126, 1070, 1032, 959, 925, 898, 842, 720, 699, 544, 472, 429 cm⁻¹.

### Example 21

### (RS)-(Z)-16-Hydroxyoctadec-14-enoic acid (Compound No. 48)

(1) Using the compound obtained in Example 10 (1), the reaction was carried out in the same manner as in Example 2 (1) to afford (RS)-(Z)-16-hydroxyoctadec-14-enoic acid ethyl ester.

¹H-NMR (CDCl₃, 300 MHz) δ ppm: 0.91 (t, J=7.5 Hz, 3H), 1.18-1.68 (m, 25H), 1.97-2.16 (m, 2H), 2.29 (t, J=7.5 Hz, 2H), 4.12 (q, J=7.2 Hz, 2H), 4.31-4.41 (m, 1H), 5.31-5.56 (m, 2H).

IR (neat): 3428, 2925, 2854, 1739, 1465, 1374, 1246, 1180, 1110, 1034, 966, 722 cm⁻¹.
(2) Using the compound obtained in the above (1), the reaction was carried out in the same manner as in Example 1 (2) to afford the title compound.

¹H-NMR (CDCl₃, 300 MHz) δ ppm: 0.91 (t, J=7.5 Hz, 3H), 1.18-1.70 (m, 22H), 1.95-2.18 (m, 2H), 2.35 (t, J=7.5 Hz, 2H), 4.31-4.41 (m, 1H), 5.29-5.70 (m, 2H).

IR (KBr): 3284, 2922, 2852, 1698, 1472, 1433, 1412, 1302, 1278, 1255, 1230, 1208, 1188, 1121, 1072, 962, 856, 793, 742, 718, 684, 529 cm⁻¹.

### Example 22

### (R)-16-Hydroxyeicos-14-ynenitrile (Compound No. 37)

(1) The reaction was carried out in the same manner as in Example 11 (1), but using 1,12-dibromododecane instead of 1,7-dibromoheptane, to afford ((R)-15-bromo-1-butylpentadec-2-ynyloxy)-tert-butyldimethylsilane.

¹H-NMR (CDCl₃, 300 MHz) δ ppm: 0.10 (s, 3H), 0.12 (s, 3H), 0.88-0.92 (m, 12H), 1.24-1.52 (m, 22H), 1.58-1.67 (m, 2H), 1.80-1.93 (m, 2H), 2.18 (dt, J=2.0, 6.9 Hz, 2H), 3.41 (t, J=6.8 Hz, 2H), 4.31 (ddt, J=1.9, 1.9, 6.5 Hz, 1H).

IR (neat): 2930, 2856, 1464, 1361, 1341, 1251, 1152, 1110, 1083, 1005, 938, 838, 778, 667, 566 cm⁻¹.
(2) To a solution of sodium cyanide (735 mg, 15 mmol) in DMSO (dimethyl sulfoxide) (20 mL) (distilled after drying) was added dropwise over 10 minutes, while heating at 80° C. with stirring, the compound obtained in the above (1) (4.74 g, 10 mmol) and then the mixture was stirred for 2 hours. The reaction solution was allowed to cool down to room temperature, poured into water, and the mixture was extracted with hexane (200 mL) and then washed with water (50 mL). It was dried over anhydrous magnesium sulfate and concentrated. The resulting crude product was purified by silica gel column chromatography to afford (R)-16-(tert-butyldimethylsilanyloxy)eicos-14-ynenitrile (3.73 g).

¹H-NMR (CDCl₃, 300 MHz) δ ppm: 0.10 (s, 3H), 0.12 (s, 3H), 0.84-0.92 (m, 12H), 1.19-1.52 (m, 22H), 1.58-1.72 (m, 4H), 2.18 (dt, J=2.0, 7.0 Hz, 2H), 2.33 (t, J=7.1 Hz, 2H), 4.27-4.36 (m, 1H).
(3) The reaction was carried out in the same manner as in Example 11 (2), but using the compound obtained in the above (2) instead of the compound obtained in Example 11 (1), to afford the title compound.

¹H-NMR (CDCl₃, 300 MHz) δ ppm: 0.92 (t, J=7.1 Hz, 3H), 1.20-1.74 (m, 26H), 2.20 (dt, J=1.9, 7.0 Hz, 2H), 2.33 (t, J=7.2 Hz, 2H), 4.28-4.39 (m, 1H).

IR (neat): 3436, 2929, 2856, 2247, 1466, 1147, 1104, 1038, 1008, 723 cm⁻¹.

### Example 23

### (R)-19-(1H-Tetrazol-5-yl)nonadec-6-yn-5-ol (Compound No. 36)

To a solution of the compound obtained in Example 22 (1.0 g, 3.3 mmol) in DMF (dimethylformamide) (30 mL) were added sodium azide (644 mg, 9.9 mmol) and ammonium chloride (530 mg, 9.9 mmol) and the mixture was heated under reflux at 125° C. for 39 hours. After completion of the reaction, the reaction solution was poured into water (100 mL), and the mixture was extracted with AcOEt (200 mL). The organic layer was washed with water (50 mL) and then brine (50 mL). It was dried over anhydrous magnesium sulfate and concentrated. The resulting crude product was purified by silica gel column chromatography and recrystallized from Et₂O/petroleum ether to afford the title compound (442 mg).

¹H-NMR (CDCl₃, 300 MHz) δ ppm: 0.92 (t, J=7.1 Hz, 3H), 1.17-1.54 (m, 22H), 1.62-1.92 (m, 4H), 2.14-2.24 (m, 2H), 2.97-3.11 (m, 2H), 4.38-4.47 (m, 1H).

IR (KBr): 3208, 2920, 2852, 1546, 1472, 1408, 1378, 1292, 1261, 1246, 1228, 1214, 1147, 1107, 1066, 1047, 1008, 825, 758, 718, 608 cm⁻¹.

### Example 24

### (R)-19-Bromononadec-6-yn-5-ol (Compound No. 109)

The reaction was carried out in the same manner as in Example 11 (2), but using the compound obtained in Example 22 (1) instead of the compound obtained in Example 11 (1), to afford the title compound.

¹H-NMR (CDCl₃, 300 MHz) δ ppm: 0.92 (t, J=7.1 Hz, 3H), 1.23-1.58 (m, 22H), 1.60-1.74 (m, 2H), 1.79-1.92 (m, 2H), 2.20 (dt, J=2.0, 7.0 Hz, 2H), 3.41 (t, J=6.8 Hz, 2H), 4.30-4.39 (m, 1H).

IR (neat): 3368, 2927, 2855, 2230, 1466, 1148, 1037, 722, 646, 563 cm⁻¹.

### Example 25

### (R)-(Z)-19-Bromononadec-6-en-5-ol (Compound No. 110)

The reaction was carried out in the same manner as in Example 2 (1), but using the compound obtained in Example 24 instead of the compound obtained in Example 1 (1), to afford the title compound.

¹H-NMR (CDCl₃, 300 MHz) δ ppm: 0.91 (t, J=6.9 Hz, 3H), 1.20-1.65 (m, 24H), 1.79-1.92 (m, 2H), 2.01-2.15 (m, 2H), 3.41 (t, J=6.8 Hz, 2H), 4.37-4.47 (m, 1H), 5.31 (m, 2H).

IR (neat): 3368, 3005, 2925, 2854, 1656, 1466, 1378, 1251, 1008, 722, 647, 564 cm⁻¹.

### Example 26

### (R)-(Z)-19-Hydroxyeicos-14-enenitrile (Compound No. 111)

The reaction was carried out in the same manner as in Example 22 (2), but using the compound obtained in Example 25 instead of the compound obtained in Example 22 (1), to afford the title compound.

¹H-NMR (CDCl₃, 300 MHz) δ ppm: 0.91 (t, J=7.0 Hz, 3H), 1.20-1.72 (m, 26H), 2.00-2.14 (m, 2H), 2.33 (t, J=7.1 Hz, 2H), 4.37-4.48 (m, 1H), 5.31-5.54 (m, 2H).

IR (neat): 3436, 2926, 2854, 2247, 1466, 1007, 723, 500 cm⁻¹.

### Example 27

### (R)-(Z)-19-(1H-Tetrazol-5-yl)nonadec-6-en-5-ol (Compound No. 59)

The reaction was carried out in the same manner as in Example 23, but using the compound obtained in Example 26 instead of the compound obtained in Example 22, to afford the title compound.

¹H-NMR (CDCl₃, 300 MHz) δ ppm: 0.90 (t, J=6.8 Hz, 3H), 1.16-1.56 (m, 23H), 1.58-1.72 (m, 1H), 1.76-1.90 (m, 2H), 1.96-2.20 (m, 2H), 3.02 (t, J=7.7 Hz, 2H), 4.46-4.58 (m, 1H), 5.34-5.58 (m, 2H).

IR (neat): 3292, 3006, 2925, 2854, 2627, 2098, 1656, 1558, 1466, 1378, 1251, 1103, 1054, 1001, 897, 724 cm⁻¹.

### Example 28

### (RS)-(Z)-15-Hydroxyoctadec-13-enoic acid (Compound No. 72)

(1) The reaction was carried out substantially in the same manner as in Example 1 (1), but using 12-bromododecanoic acid and (RS)-3-tert-butyldimethylsiloxy-1-hexyne instead of 13-bromotridecanoic acid and (R)-3-tert-butyldimethylsiloxy-1-heptyne, respectively, to afford (RS)-15-hydroxyoctadecs-13-ynoic acid ethyl ester.

¹H-NMR (CDCl₃, 300 MHz) δ ppm: 0.95 (t, J=7.2 Hz, 3H), 1.21-1.74 (m, 25H), 2.20 (dt, J=1.9, 7.0 Hz, 2H), 2.29 (t, J=7.5 Hz, 2H), 4.13 (q, J=7.2 Hz, 2H), 4.32-4.40 (m, 1H).

IR (neat): 3436, 2929, 2855, 1737, 1466, 1374, 1248, 1180, 1100, 1029, 854, 723 cm⁻¹.
(2) Using the compound obtained in the above (1), the reaction was carried out in the same manner as in Example 2 (1) to afford (RS)-(Z)-15-hydroxyoctadec-13-enoic acid ethyl ester.

¹H-NMR (CDCl₃, 300 MHz) δ ppm;0.93 (t, J=7.2 Hz, 3H), 1.20-1.68 (m, 25H), 2.02-2.13 (m, 2H), 2.28 (t, J=7.5 Hz, 2H), 4.12 (q, J=7.2 Hz, 2H), 5.31-5.40 (m, 1H), 5.43-5.54 (m, 1H).

IR (neat): 3428, 2926, 2854, 2360, 1739, 1466, 1374, 1350, 1247, 1180, 1098, 1063, 1033, 848, 723 cm⁻¹.
(3) Using the compound obtained in the above (2), the reaction was carried out in the same manner as in Example 1 (2) to afford the title compound.

¹H-NMR (CDCl₃, 300 MHz) δ ppm: 0.93 (t, J=7.2 Hz, 3H), 1.20-1.75 (m, 22H), 1.93-2.20 (m, 2H), 2.35 (t, J=7.5 Hz, 2H), 4.40-4.49 (m, 1H), 5.36 (dt, J=8.7, 1.4 Hz, 1H), 5.43-5.53 (m, 1H).

IR (neat): 3368, 2926, 2854, 1711, 1466, 1384, 1247, 1098, 1064, 1004, 756 cm⁻¹.

### Example 29

### (S)-(Z)-16-Hydroxyeicos-14-enoic acid (Compound No. 56)

(1) To the compound obtained in Example 2 (1) (250 mg, 0.71 mmol) were added benzoic acid (130 mg, 1.06 mmol) triphenylphosphine (278 mg, 1.06 mmol) and diethyl azodicarboxylate (0.46 ml, 1.06 mmol) at 0° C. under argon stream and then the mixture was stirred for one hour while allowing to rise up to room temperature. To the reaction solution was added hexane (5 mL), the mixture was filtered and purified by silica gel column chromatography to afford benzoic acid (S)-(Z)-1-butyl-15-ethoxycarbonylpentadec-2-enyl ester (149 mg).

¹H-NMR (CDCl₃, 300 MHz) δ ppm: 0.91 (t, J=7.1 Hz, 3H), 1.19-1.43 (m, 25H), 1.50-1.86 (m, 4H), 2.13-2.33 (m, 4H), 4.12 (q, J=7.1 Hz, 2H), 5.36-5.48 (m, 1H), 5.50-5.63 (m, 1H), 5.72-5.83 (m, 1H), 7.38-7.47 (m, 2H), 7.50-7.57 (m, 1H), 7.99-8.07 (m, 2H).

IR (neat): 2928, 2855, 1736, 1718, 1603, 1585, 1466, 1452, 1372, 1315, 1271, 1177, 1110, 1070, 1027, 945, 712, 688 cm⁻¹.
(2) To a solution of the compound obtained in the above (1) (149 mg, 0.325 mmol) in EtOH (1 mL) was added a 20% solution of sodium ethoxide in ethanol (0.17 mL, 0.488 mmol) and the mixture was stirred at room temperature overnight. The reaction solution thus obtained was poured into a saturated ammonium chloride solution (10 mL), extracted with ethyl acetate (20 mL×2) and the organic layer was washed with brine (30 ml) and dried over anhydrous magnesium sulfate. The resulting crude product was purified by silica gel column chromatography to afford (S)-(Z)-16-hydroxyeicos-14-enoic acid ethyl ester (53 mg).

¹H-NMR (CDCl₃, 300 MHz) δ ppm; 0.90 (t, J=6.6 Hz, 3H), 1.21-1.68 (m, 29H), 2.02-2.12 (m, 2H), 2.28 (t, J=7.5 Hz, 2H), 4.12 (q, J=7.1 Hz, 2H), 4.37-4.49 (m, 1H), 5.31-5.40 (m, 1H), 5.43-5.57 (m, 1H).

IR (neat): 3428, 2926, 2855, 1739, 1466, 1375, 1180, 1100, 1031, 723 cm⁻¹.
(3) Using the compound obtained in the above (2) (48 mg, 0.135 mmol), the reaction was carried out in the same manner as in Example 1 (2) to afford the title compound (40 mg.).

¹H-NMR (CDCl₃, 300 MHz) δ ppm: 0.90 (t, J=6.8 Hz, 3H), 1.20-1.51 (m, 22H), 1.54-1.70 (m, 4H), 2.00-2.16 (m, 2H), 2.35 (t, J=7.5 Hz, 2H), 4.43 (dt, J=8.5, 6.4 Hz, 1H), 5.31-5.41 (m, 1H), 5.43-5.55 (m, 1H).

IR (KBr): 3277, 2922, 2852, 1703, 1468, 1438, 1302, 1105, 1047, 1017, 972, 791, 721, 638, 466 cm⁻¹.

### Example 30

### (R)-(Z)-(13-Hydroxyheptadec-11-enyloxy) acetic acid (Compound No. 102)

(1) To a solution of prop-2-ynyloxyacetic acid (1.14 g, 10 mmol) in a mixed solvent of THF (10 mL) and HMPA(5 mL) was added dropwise n-BuLi (9.23 mL, 2.46M in hexane, 24 mmol) at −50° C. under argon stream. Thereafter, temperature was allowed to rise up to −30° C. over 30 minutes and then to the reaction solution thus obtained was added dropwise a solution of 2-(7-bromoheptyloxy)-tetrahydropyran (4.19 g, 15 mmol) in THF (10 mL). After allowed to rise up to room temperature over 2 hours with stirring, the reaction solution was made to acidic by the addition of an aqueous hydrochloric acid (3.0M) and extracted with AcOEt (60 mL×2). The organic layer was washed with brine (100 mL). The organic layer was dried over anhydrous magnesium sulfate and the solvent was distilled off under reduced pressure. To a solution of the crude product thus obtained in ethanol (50 mL) was added conc. sulfuric acid (0.5 mL) and the resulting mixture was stirred at room temperature overnight. The reaction solution was poured into a saturated aqueous sodium bicarbonate solution and extracted with ethyl acetate (100 mL×2). The organic layer was washed with brine, dried over anhydrous magnesium sulfate, and concentrated. The resulting crude product was purified by silica gel column chromatography to afford (10-hydroxydec-2-ynyloxy)acetic acid ethyl ester (0.92 g).

¹H-NMR (CDCl₃, 300 MHz) δ ppm: 1.20-1.64 (m, 13H), 2.22 (tt, J=7.0, 2.2 Hz, 2H), 3.65 (t, J=6.5 Hz, 2H), 4.18 (s, 2H), 4.24 (q, J=7.2 Hz, 2H), 4.29 (t, J=2.2 Hz, 2H).

IR (Neat): 3400, 2933, 2858, 2221, 1752, 1639, 1450, 1384, 1278, 1208, 1137, 1114, 1027, 936, 858, 722, 595, 500 cm⁻¹.
(2) To a solution of the compound obtained in the above (1) (0.92 g, 3.59 mmol) and carbon tetrabromide (1.55 g, 4.7 mmol) in dichloromethane (30 ml) was added a solution of triphenylphosphine (1.32 g, 4.7 mmol) in dichloromethane (10 mL) under ice-cooling and argon stream. After stirring for one hour, the dichloromethane was distilled off under reduced pressure and then the crude product was purified by silica gel column chromatography to afford 10-bromodec-2-ynyloxy)acetic acid ethyl ester (1.05 g).

¹H-NMR (CDCl₃, 300 MHz) δ ppm: 1.22-1.58 (m, 11H), 1.81-1.93 (m, 2H), 2.22 (tt, J=7.0, 2.2 Hz, 2H), 3.41 (t, J=6.8 Hz, 2H), 4.17 (s, 2H), 4.24 (q, J=7.2 Hz, 2H), 4.29 (t, J=2.2 Hz, 2H).

IR (neat): 2934, 2858, 2220, 1752, 1450, 1380, 1249, 1205, 1138, 1113, 1028, 937, 859, 723, 644, 561 cm⁻¹.
(3) To a solution of the compound obtained in the above (2) (1.0 g, 3.13 mmol) in ethanol (20 ml) was added Pd—C(5%, 50 mg) and the mixture was stirred at room temperature for one hour under hydrogen gas atmosphere. The reaction solution was filtered through Celite and concentrated, and the crude product was purified by silica gel column chromatography to afford (10-bromodecyloxy)acetic acid ethyl ester (0.76 g).

¹H-NMR (CDCl₃, 300 MHz) δ ppm: 1.17-1.49 (m, 15H), 1.54-1.69 (m, 2H), 1.79-1.92 (m, 2H), 3.41 (t, J=6.9 Hz, 2H), 3.52 (t, J=6.7 Hz, 2H), 4.06 (s, 2H), 4.22 (q, J=7.2 Hz, 2H).

IR (neat): 2929, 2855, 1757, 1736, 1466, 1376, 1273, 1201, 1139, 1032, 723, 646, 564 cm⁻¹.
(4) To a solution of the compound obtained in the above (3) in THF (30 mL) was added an aqueous solution of sodium hydroxide (8.9 mL, 1.0M) and the resulting mixture was stirred at 30° C. for 3 days. The reaction solution was poured into a saturated aqueous ammonium chloride solution and extracted with ethyl acetate (75 mL×2). The organic layer was washed with brine, dried over anhydrous sodium sulfate and concentrated. The resulting crude product was purified by silica gel column chromatography to afford (10-bromodecyloxy)acetic acid (415 mg).

¹H-NMR (CDCl₃, 300 MHz) δ ppm: 1.22-1.50 (m, 12H), 1.57-1.70 (m, 2H), 1.80-1.92 (m, 2H), 3.41 (t, J=6.8 Hz, 2H), 3.58 (t, J=6.7 Hz, 2H), 4.09 (s, 2H).

IR (neat): 2928, 2855, 2284, 1734, 1431, 1245, 1134, 723, 677, 562 cm⁻¹.
(5) The reaction was carried out substantially in the same manner as in Example 1 (1), but using the compound obtained in the above (4) instead of 13-bromotridecanoic acid to afford (R)-(13-hydroxyheptadec-11-ynyloxy)acetic acid ethyl ester.

¹H-NMR (CDCl₃, 300 MHz) δ ppm: 0.92 (t, J=7.1 Hz, 3H), 1.20-1.75 (m, 25H), 2.20 (dt, J=2.0, 7.0 Hz, 2H), 3.52 (t, J=6.7 Hz, 2H), 4.06 (s, 2H), 4.22 (q, J=7.1 Hz, 2H), 4.30-4.39 (m, 1H).

IR (neat): 3468, 2930, 2857, 1756, 1466, 1377, 1275, 1202, 1138, 1034, 723 cm⁻¹.
(6) The reaction was carried out substantially in the same manner as in Example 2 (1), but using the compound obtained in the above (5) to afford (R)-(Z)-(13-hydroxyheptadec-11-enyloxy)acetic acid ethyl ester.

¹H-NMR (CDCl₃, 300 MHz) δ ppm: 0.90 (t, J=6.8 Hz, 3H), 1.20-1.70 (m, 25H), 2.00-2.15 (m, 2H), 3.52 (t, J=6.7 Hz, 2H), 4.06 (s, 2H), 4.22 (q, J=7.2 Hz, 2H), 4.38-4.47 (m, 1H), 5.32-5.41 (m, 1H), 5.43-5.53 (m, 1H).

IR (neat): 3436, 2927, 2855, 2361, 1757, 1656, 1466, 1377, 1275, 1202, 1139, 1027, 723 cm⁻¹.
(7) The reaction was carried out substantially in the same manner as in Example 1 (2), but using the compound obtained in the above (6) to afford the title compound.

¹H-NMR (CDCl₃, 300 MHz) δ ppm: 0.90 (t, J=6.9 Hz, 3H), 1.18-1.50 (m, 22H), 1.54-1.69 (m, 2H), 1.99-2.17 (m, 2H), 3.57 (t, J=6.6 Hz, 2H), 4.09 (s, 2H), 4.39-4.49 (m, 1H), 5.32-5.41 (m, 1H), 5.44-5.45 (m, 1H).

IR (neat): 3400, 2927, 2855, 2361, 1734, 1466, 1384, 1240, 1136, 1021, 756, 670, 571 cm⁻¹.

### Example 31

### (R)-5-(14-Hydroxyoctadec-12-ynyl)thiazolidine-2,4-dione (Compound No. 33)

(1) To a solution of (R)-tert-butyldimetylsiloxy-1-heptyne (3.01 g, 13.3 mmol) in THF (20 mL) was added dropwise under argon stream n-BuLi (5.95 mL, 2.46M in hexane, 14.6 mmol) at 0° C. and then the reaction solution was cooled to −40° C. and then added dropwise to 1,11-dibromo undecane (6.87 g, 21.9 mmol) in a mixed solvent of THF (50 mL) and DMPU (20 ml). The reaction solution was allowed to rise up to room temperature over 1.5 hours. To the resulting solution was added an aqueous hydrochloric acid (10 mL, 3.0M) and the mixture was extracted with hexane (100 mL×2). The organic layer was washed with brine (200 mL), dried over anhydrous magnesium sulfates concentrated. The resulting crude product was purified by distillation to afford (R)-(14-bromo-1-butyltetradec-2-ynyloxy)-tert-butyldimethylsilane (3.39 g).

¹H-NMR (CDCl₃, 300 MHz) δ ppm: 0.10 (s, 3H), 0.12 (s, 3H), 0.84-0.96 (m, 12H), 1.20-1.68 (m, 26H), 1.80-1.91 (m, 2H), 2.18 (dt, J=1.9, 6.9 Hz, 2H), 3.41 (t, J=6.8 Hz, 2H), 4.27-4.35 (m, 1H).

IR (neat): 2929, 2856, 1464, 1361, 1341, 1251, 1110, 1083, 1006, 938, 837, 778, 667, 565 cm⁻¹.
(2) To a solution of 2,4-thiazolidinedione (141 mg, 1.2 mmol) in a mixed solvent of THF (5 mL) and HMPA(3 mL) under a stream of argon was added dropwise at −60° C. n-BuLi (1.17 ml, 2.46M in hexane, 2.88 mmol). The mixture was stirred at that temperature for 30 minutes and then at room temperature for a further 30 minutes. The mixture was again cooled to −60° C., a solution of the compound obtained in the above (1) (460 mg, 1.0 mmol) in THF (5 mL) was added dropwise and then the mixture was allowed to rise up to 0° C. over 4 hours. To the resulting solution was added an aqueous hydrochloric acid (5 mL, 3.0M) and the mixture was extracted with hexane (100 mL×2). The organic layer was washed with brine (200 mL), dried over anhydrous magnesium sulfate and concentrated. The resulting crude product was purified by silica gel column chromatography to afford (R)-5-[14-(tert-butyldimethylsiloxy)octadec-12-ynyl]thiazolidine-2,4-dione (175 mg).

¹H-NMR (CDCl₃, 300 MHz) δ ppm: 0.10 (s, 3H), 0.12 (s, 3H), 0.84-0.96 (m, 12H), 1.20-1.68 (m, 26H), 2.18 (dt, J=2.0, 6.9 Hz, 2H), 4.24-4.36 (m, 2H), 7.26 (bs, 1H).

IR (neat): 3216, 3067, 2928, 2855, 2231, 1758, 1702, 1464, 1385, 1333, 1250, 1152, 1110, 1084, 1005, 937, 837, 777, 668, 605, 536 cm⁻¹.
(3) To a solution of the compound obtained in the above (2) (170 mg, 0.35 mmol) in MeOH (5 mL) was added an aqueous hydrochloric acid (0.5 mL, 3.0M) and the mixture was stirred at room temperature for one hour. The solution was poured into a saturated aqueous sodium bicarbonate solution (5 mL) and then extracted with ethyl acetate (20 mL×2). The organic layer was washed with brine (30 mL), dried over anhydrous magnesium sulfate and concentrated. The resulting crude product was purified by silica gel column chromatography to afford the title compound (104 mg).

¹H-NMR (CDCl₃, 300 MHz) δ ppm: 0.92 (t, J=7.1 Hz, 3H), 1.18-2.24 (m, 28H), 4.27 (dd, J=9.2, 4.3 Hz, 1H), 4.35 (ddt, J=1.9, 1.9, 6.6 Hz, 1H), 8.56 (bs, 1H).

IR (neat): 3346, 3160, 3053, 2921, 2850, 2229, 1753, 1724, 1468, 1329, 1209, 1164, 1107, 1046, 889, 774, 739, 722, 671, 610, 546, 465, 428 cm⁻¹.

### Example 32

### (R)-(Z)-5-(14-Hydroxyoctadec-12-enyl)thiazolidine-2,4-dione (Compound No. 68)

(1) To a solution of the compound obtained in Example 31 (1) (4.28 g, 9.31 mmol) in MeOH (50 mL) was added an aqueous hydrochloric acid (0.5 ml, 3.0M) and the mixture was stirred at room temperature for one hour. The reaction solution was poured into a saturated aqueous sodium bicarbonate solution (10 mL) and extracted with ethyl acetate (30 mL×2). The organic layer was washed with brine (50 mL), dried over anhydrous magnesium sulfate and concentrated. The resulting crude product was purified by silica gel column chromatography to afford (R)-18-bromooctadec-6-yn-5-ol (1.59 g).

¹H-NMR (CDCl₃, 300 MHz) δ ppm: 0.92 (t, J=7.1 Hz, 3H), 1.21-1.57 (m, 20H), 1.60-1.74 (m, 2H), 1.80-1.92 (m, 2H), 2.20 (dt, J=2.0, 7.0 Hz, 2H), 3.41 (t, J=6.9 Hz, 2H), 4.30-4.40 (m, 1H).

IR (neat): 3368, 2929, 2855, 2215, 1672, 1466, 1384, 1148, 1039, 723, 646, 564 cm⁻¹.
(2) The reaction was carried out substantially in the same manner as in Example 2 (1), but using the compound obtained in the above (1) to afford (R)-(Z)-18-bromooctadec-6-en-5-ol.

¹H-NMR (CDCl₃, 300 MHz) δ ppm: 0.91 (t, J=6.9 Hz, 3H), 1.20-1.68 (m, 22H), 1.80-1.92 (m, 2H), 1.99-2.15 (m, 2H), 3.41 (t, J=6.9 Hz, 2H), 4.38-4.48 (m, 1H), 5.32-5.42 (m, 1H), 5.43-5.54 (m, 1H).

IR (neat): 3368, 3005, 2926, 2854, 1466, 1378, 1251, 1008, 723, 646, 564 cm⁻¹.
(3) To a solution of the compound obtained in the above (2) (500 mg, 1.38 mmol) in DMF(25 mL) were added tert-butyldimethylsilyl chloride (230 mg, 1.52 mmol) and imidazole (188 mg, 2.76 mmol). The mixture was stirred at room temperature overnight. The reaction solution was poured into a saturated aqueous sodium bicarbonate solution (10 mL), extracted with ethyl acetate (30 mL×2). The organic layer was washed with brine (50 mL). The organic layer was washed with brine (50 mL), dried over anhydrous magnesium sulfate and concentrated. The resulting crude product was purified by silica gel column chromatography to afford (R)-(Z)-(14-bromo-1-butyltetradec-2-enyloxy)-tert-butyldimethylsilane (650 mg).

¹H-NMR (CDCl₃, 300 MHz) δ ppm: 0.02 (s, 3H), 0.04 (s, 3H), 0.81-0.94 (m, 12H), 1.18-1.60 (m, 22H), 1.71-1.82 (m, 2H), 1.94-2.09 (m, 2H), 3.53 (t, J=6.8 Hz, 2H), 4.33-4.43 (m, 1H), 5.29-5.37 (m, 2H).

IR (neat): 2956, 2928, 2856, 1464, 1361, 1253, 1078, 1006, 939, 836, 775, 723, 668 cm⁻¹.
(4) Using the compound obtained in the above (3), the reaction was carried out in the same manner as in Example 31 (2) to afford 5-[(R)-(Z)-14-(tert-butyldimethylsiloxy)octadec-12-enyl]thiazolidine-2,4-dione.

¹H-NMR (CDCl₃, 300 MHz) δ ppm: 0.02 (s, 3H), 0.04 (s, 3H), 0.89-0.96 (m, 12H), 1.20-1.62 (m, 22H), 1.84-2.08 (m, 4H), 2.09-2.25 (m, 2H), 4.28 (dd, J=9.2, 4.2 Hz, 1H), 4.33-4.43 (m, 1H), 5.24-5.37 (m, 2H), 7.88 (bs, 1H).

IR (neat): 3216, 3011, 2927, 2855, 1758, 1702, 1464, 1385, 1361, 1330, 1253, 1152, 1006, 939, 836, 775, 669, 605, 536 cm⁻¹.
(5) Using the compound obtained in the above (4), the reaction was carried out in the same manner as in Example 31 (3) to afford the title compound.

¹H-NMR (CDCl₃, 300 MHz) δ ppm: 0.90 (t, J=6.8 Hz, 3H), 1.20-1.71 (m, 24H), 1.84-2.24 (m, 4H), 4.27 (dd, J=9.0, 4.2 Hz, 1H), 4.38-4.48 (m, 1H), 5.31-5.54 (m, 1H), 5.43-5.54 (m, 1H), 8.51 (bs, 1H).

IR (KBr): 3348, 3160, 3060, 2921, 2850, 1753, 1720, 1656, 1561, 1542, 1509, 1468, 1330, 1212, 1164, 1054, 739, 671, 610, 546, 466, 438 cm⁻¹.

### Example 33

### N-((R)-(Z)-16-Hydroxyeicos-14-enoyl)-4-methylbenzenesulfonamide (Compound No. 53)

(1) To a solution of the compound obtained in Example 2 (150 mg, 0.46 mmol) in THF (5 mL) were added N-hydroxysuccinimide (159 mg, 1.38 mmol) and 1-(3-dimethylaminopropyl)-3-ethylcarbodiimide hydrochloride (265 mg, 1.38 mmol) at 0° C. The mixture was stirred at that temperature for 2 days. To the reaction solution was added water (50 mL) and the mixture was extracted with ethyl acetate (50 mL×2). The organic layer was washed with brine (100 mL), dried over anhydrous magnesium sulfate and concentrated. The resulting crude product was purified by silica gel chromatography to afford (R)-(Z)-16-hydroxyeicos-14-enoic acid 2,5-dioxopyrrolidin-1-yl ester (163 mg).

¹H-NMR (CDCl₃, 300 MHz) δ ppm: 0.90 (t, J=6.8 Hz, 3H), 1.18-1.80 (m, 26H), 2.02-2.15 (m, 2H), 2.60 (t, J=7.5 Hz, 2H), 2.83 (s, 4H), 4.38-4.48 (m, 1H), 5.31-5.41 (m, 1H), 5.43-5.53 (m, 1H).

IR (KBr): 3349, 2923, 2853, 1827, 1790, 1728, 1470, 1407, 1381, 1211, 1150, 1072, 996, 869, 814, 722, 655, 582, 553, 420 cm⁻¹.
(2) To a solution of the compound obtained in the above (1) (70 mg, 0.165 mmol) in THF (3 mL) were added p-toluenesulfonamide (283 mg, 1.65 mmol) and DBU (1,8-diazabicyclo[5.4.0]undec-7-ene) (0.027mL, 0.182mmol) and the mixture was stirred at room temperature overnight. The reaction solution was poured into a saturated ammonium chloride solution (30 mL) and extracted with ethyl acetate (50 mL×2). The organic layer was washed with brine (50 mL), dried over anhydrous magnesium sulfate and concentrated. The resulting crude product was purified by silica gel column chromatography to afford the title compound (36 mg).

¹H-NMR (CDCl₃, 300 MHz) δ ppm: 0.90 (t, J=6.9 Hz, 3H), 1.16-1.66 (m, 26H), 2.00-2.14 (m, 2H), 2.22 (t, J=7.5 Hz, 2H), 2.44 (s, 3H), 4.38-4.48 (m, 1H), 5.32-5.42 (m, 1H), 5.44-5.54 (m, 1H), 7.30-7.37 (m, 2H), 7.90-8.00 (m, 3H).

IR (KBr): 3311, 3008, 2927, 2852, 1726, 1598, 1472, 1427, 1410, 1387, 1337, 1305, 1188, 1174, 1124, 1085, 1068, 1022, 1004, 861, 850, 816, 720, 671, 550 cm⁻¹.

### Example 34

### (R)-(Z)-16-Hydroxyeicos-14-enoic acid hydroxyamide (Compound No. 55)

To a solution of the compound obtained in Example 2 (80 mg, 0.245 mmol) in Et₂O (2 mL) were added ethyl chloroformate (28 μL, 0.294 mmol) and N-methylmorpholine (35 μL, 0.319 mmol) at 0° C. The mixture was stirred at that temperature for 30 minutes. Then, the reaction solution was filtered and to the filtrate was added salt-free hydroxylamine (60 mg), and the resulting mixture was stirred at room temperature for 30 minutes and concentrated. The resulting crude product was purified by silica gel column chromatography to afford the title compound (12 mg).

¹H-NMR (CDCl₃, 300 MHz) δ ppm: 0.90 (t, J=6.8 Hz, 3H), 1.15-1.75 (m, 26H), 1.96-2.28 (m, 4H), 4.38-4.48 (m, 1H), 5.31-5.42 (m, 1H), 5.44-5.54 (m, 1H).

IR (neat): 3255, 2917, 2848, 2286, 1656, 1467, 1384, 1076, 722, 503 cm⁻¹.

### Example 35

### (R)-(Z)-16-Hydroxyeicos-14-enoic acid (2-hydroxyethyl)amide (Compound No. 52)

To a solution of the compound obtained in Example 2 (300 mg, 0.92 mmol) in CH₂Cl₂ (10 mL) under argon stream was added dropwise oxalyl chloride (1.01 mL, 2M in CH₂Cl₂, 2.02 mmol) at room temperature and the mixture was stirred for 2 hours. The reaction solution was distilled under reduced pressure. The residue thus obtained was dissolved in CH₂Cl₂ (10 mL), ethanol amine (0.45 mL, 7.36 mmol) was added and then the mixture was stirred at room temperature for 2 hours. To the solution was added water and extracted with Et₂O (50 mL×2). The organic layer was washed with brine (50 mL). The organic layer was dried over anhydrous magnesium sulfate, and concentrated. The resulting crude product was purified by silica gel column chromatography to afford the title compound (132 mg).

¹H-NMR (CDCl₃, 300 MHz) δ ppm: 0.90 (t, J=6.9 Hz, 3H), 1.21-1.72 (m, 26H), 2.00-2.15 (m, 2H), 2.21 (t, J=7.7 Hz, 2H), 3.39-3.47 (m, 2H), 3.70-3.76 (m, 2H), 4.37-4.38 (m, 1H), 5.32-5.41 (m, 2H), 5.43-5.54 (m, 1H), 5.89 (bs, 1H).

IR (KBr): 3296, 3089, 3014, 2920, 2851, 1642, 1555, 1464, 1441, 1379, 1319, 1280, 1216, 1181, 1126, 1060, 1040, 1004, 876, 730, 688, 610, 540 cm⁻¹.

### Test Example 1

### Test for Elastase Production by fMLP (N-formyl-Met-Leu-Phe) Stimulation

Rat neutrophils preparation was obtained 15-18 hrs after intraperitoneal injection of a 1% sterile casein solution in saline (120 mL/kg). Cells were harvested by peritoneal lavage after the decapitation. The lavage fluid was ice-cold PBS (Phosphate-Buffered Saline). Peritoneal exudates were pooled, centrifuged and suspended in HBSS (Hanks' Balanced Salt Solution) at 1×10⁷ cells/mil. Cytochalasin B (final concentration: 5 μg/ml) were added to prime the cells. The cells were added into a 96-well culture plate (190 μL/well) and then the compounds of the present invention at various concentrations (10⁻⁷ to 3×10⁻⁵ M) were added and incubated at 37° C. in an atmosphere of 5% CO₂ in air. After 10 minutes, fMLP (20 μM, 10 μL) was added, while 10 μL of an HBSS solution containing 0.4% ethanol was added to the group to which no fMLP was added. After gently stirring, cells were incubated for further 10 minutes. The reaction was stopped on ice, and an incubated supernatant was recovered by centrifugation.

### Assay of Elastase Activity in an Incubated Supernatant

Elastase activity in the incubated supernatant was measured using a specific elastase substrate, N-succinyl-L-alanyl-L-alanyl-L-proline-valine-MCA (Peptide Institute, Inc., Osaka), 0.12 mM in 50 mM Tris-HCl (pH 8.0). Fifty microliter of an incubated supernatant was added to the substrate solution (50 μL) and incubated at 37° C. for 30 minutes. Elastase activity was assayed at a wavelength of 360 nm at excitation and 480 nm at emission.

Elastase release-inhibiting activity (inhibition ratio) was calculated according to the following equation:
Inhibition ratio (%)={1−(A−C)/(B−C)}×100
wherein A stands for a fluorescence intensity when fMLP (1 μM) was added; B stands for a fluorescence intensity when fMLP (1 μm) and the present compound were added; and C stands for a fluorescence intensity when fMLP (1 μM) was not added.

Inhibitory concentration of 50% (IC₅₀ value) of the compound of the invention was calculated with a concentration-inhibition ratio curve. The results are shown in Table 1.

TABLE 1

| Test compound | IC₅₀ value (μM) |
|---|---|
| Compound 12 | 9.18 |
| Compound 4 | 10.3 |
| Compound 50 | 8.29 |
| Compound 65 | 17.5 |

In the above Table, Compounds 12, 4, 50 and 65 correspond to the compounds of the Examples. The above results demonstrate that the compound of the present invention has a potent inhibiting activity in elastase production.

### Test Example 2

### Effect of a Compound 50 on the Infarct Volume in Rat Transient MCA Occlusion (t-MCAo) Model

Methods

Adult male Wistar rats (200-250 g) were anesthetized with 2% halothane in air. The right internal carotid artery (ICA) was carefully dissected. A silicon-coated suture (18 mm-long) was inserted to the ICA. Body temperature was maintained at 37° C. with a heating pad. After surgery, anesthesia was discontinued, and ischemic animal exhibited severe hemiparesis in the upper extremities. After 1 hour of MCA occlusion, the thread was removed to allow reperfusion of the ischemic area. Rats were received intravenously 1 hour-infusion of vehicle (10% of HP-β-CD) or the compound 50 dissolved in vehicle immediately after reperfusion.

To measure infarct volume, rats were killed at 71 hours of reperfusion. Brains were perfused transcardially with physiological saline, and removed from skulls, cut into 2-mm coronal sections. The slices were immersed in 2% triphenyltetrazolium chloride (TTC) solution at 37° C. for 30 minutes.

All values were presented as mean ±SEM. For statistical analyses, Dunnett's multiple-range test was used.

### Results

### Dose-Dependent Effect of the Compound 50 on Infarct Volume in Rat Transient MCAo Model

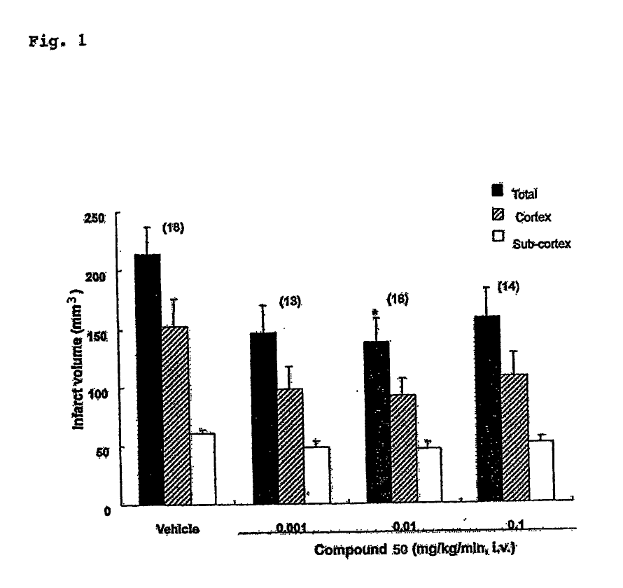

The compound 0.001, 0.01 and 0.1 mg/kg/min dissolved in 10% of HP-β-CD were continuously administrated for 1 hour from immediately after reperfusion. The compound reduced the infarct volume from 0.001 mg/kg/min, and significantly reduced the total infract volume by 35.3% as compared with vehicle-treated group at a dose of 0.01 mg/kg/min (FIG. 1). This result indicates that the compound 50 has also protective effect against ischemic brain damage.

### Industrial Applicability

The hydroxyeicosenoic acid analog according to the invention has a potent elastase release-inhibiting activity and it is then useful as an elastase release inhibitor.

Elastase is known to be involved in pathology of certain diseases such as pulmonary emphysema, respiratory distress syndrome of adults, idiopathic pulmonary fibrosis, cystic pulmonary fibrosis, chronic interstitial pneumonia, chronic bronchitis, chronic sinopulmonary infection, diffuse panbronchiolitis, bronchiectasis, asthma, pancreatitis, nephritis, hepatic insufficiency, chronic rheumatism, arthrosclerosis, osteoarthritis, psoriasis, periodontitis, atherosclerosis, rejection against organ transplantation, premature amniorrhexis, hydroa, shock, sepsis, systemic lupus erythematosus, Crohn's disease, disseminated intravenous coagulation, cerebral infarction, cardiac disorders, ischemic reperfusion disorders observed in renal diseases, cicatrization of corneal tissues, spondylitis, and etc.

The elastase release inhibitor according to the invention is therefore useful as a therapeutic or preventive agent for the above-mentioned diseases.

1. A hydroxyeicosenoic acid analog represented by the following Formula (I) 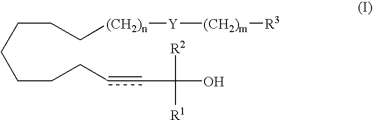
the bond [symbol] represents a cis-vinylene group or an ethynylene group;
Y represents CH₂, O or S(O)ₚ wherein p is 0, 1 or 2;
m represents an integer of 1 to 4 inclusive;
n represents an integer of 0 to 3 inclusive;
the sum of m and n is an integer of 3 to 7 inclusive;
R¹ represents a C₁₋₄ alkyl group or a C₃₋₈ cycloalkyl group;
R² represents a hydrogen atom or a methyl group;
R³ represents COR⁴, a nitrile group, a halogen atom, a tetrazole group or a thiazolidinedione group;
R⁴ represents OR⁶, NHR⁶, N(OH)R⁶, NHSO₂R⁵, glycerol or functionalized glycerols;
R⁵ represents a C₁₋₁₅ alkyl group, a C₆₋₁₀ aryl group or a C₇₋₁₄ aryl group substituted with alkyl groups, halogens or amino groups;
R⁶ represents a hydrogen atom, a C₁₋₁₀ alkyl group or a C₁₋₁₀ alkyl group substituted with a hydroxyl group, or a pharmaceutically acceptable salt or hydrate thereof.

2. The hydroxyeicosenoic acid analog of Formula (I) according to claim 1 wherein the sum of m and n is 3, 4 or 5, R¹ is a C₁₋₄ alkyl, R² is a hydrogen, R³ is COR⁴, tetrazole group or thiazolidinedione group and Y is CH₂.

3. The hydroxyeiconoic acid analog of Formula (I) according to claim 1 wherein the compound is (R)-16-Hydroxyeicos-14-ynoic acid, (R)-17-Hydroxyheneicos-15-ynoic acid, (R)-(Z)-16-Hydroxyeicos-14-enoic acid or (R)-(Z)-15-Hydroxynonadec-13-enoic acid.

4. (canceled)

5. A method for treatment of a disease in which elastase is involved, in a patient in need of such treatment, the method comprising administering to the patient, an elastase release-inhibiting amount of the hydroxyeicosenoic acid analog according to claim 1.
